(19) United States
(12) Patent Application Publication
(10) Pub. No.: US 2018/0179571 A1
(43) Pub. Date: Jun. 28, 2018

(54) PROCESSING BIOMASS

(71) Applicant: Xyleco, Inc., Wakefield, MA (US)

(72) Inventors: Marshall MEDOFF, Wakefield, MA (US); Thomas Craig MASTERMAN, Rockport, MA (US)

(21) Appl. No.: 15/904,106

(22) Filed: Feb. 23, 2018

Related U.S. Application Data
(63) Continuation of application No. 14/296,622, filed on Jun. 5, 2014.
Application No. 14/296,622 is a continuation of application No. 13/396,369, filed on Feb. 14, 2012, now abandoned.
(60) Provisional application No. 61/442,781, filed on Feb. 14, 2011.

(51) Int. Cl. C12P 21/00; C12P 19/00; C12P 7/64; C12P 7/56; C08B 1/00; C10L 5/44; C12P 19/14; C12P 19/02; C12P 7/10; C12N 1/22

(52) U.S. Cl. CPC C12P 21/00; C12P 2201/00; C12P 19/00; C12P 7/64; C12P 7/56; C08B 1/003; Y02E 50/30; Y02E 50/16; Y02E 50/10; C12P 19/14; C12P 19/02; C12P 7/10; C12N 1/22; C10L 5/44

(57) ABSTRACT

Feedstocks, obtained at least in part from a plant material that has been modified with respect to its wild type, are processed to produce useful intermediates and products, such as energy, fuels, foods or materials. For example, systems are described that can treat such feedstock materials, e.g., to reduce the recalcitrance of the feedstock, and use the treated feedstock materials to produce an intermediate or product, e.g., by saccharification and/or fermentation.

### RELATED APPLICATIONS

This application is a continuation of U.S. application Ser. No. 14/296,622, filed Jun. 5, 2014, which is a continuation of U.S. application Ser. No. 13/396,369, filed Feb. 14, 2012, which claims the benefit of and priority to U.S. Provisional Application Ser. No. 61/442,781, filed Feb. 14, 2011. The complete disclosure of these applications are hereby incorporated by reference herein.

### BACKGROUND

Cellulosic and lignocellulosic materials are produced, processed, and used in large quantities in a number of applications. Often such materials are used once, and then discarded as waste, or are simply considered to be waste materials, e.g., bagasse, sawdust, and stover. In some cases, cellulosic and lignocellulosic materials are obtained by growing and harvesting plants.

### SUMMARY

Generally, this invention relates to using and/or processing feedstock materials e.g., cellulosic and/or lignocellulosic feedstock materials, including plants that have been modified with respect to their wild types, e.g., genetically modified plants, and to intermediates and products made therefrom. Many of the methods described herein provide materials that can be more readily utilized by a variety of microorganisms to produce useful intermediates and products, e.g., energy, a fuel, a food or a material.

In one aspect, the invention features methods for making products that include physically treating a cellulosic, lignocellulosic and/or starchy feedstock obtained at least in part from a plant that has been modified with respect to a wild type variety of the plant e.g., the plant has been genetically modified. In some embodiments the entire plant can be used. In certain embodiments, a portion of the plant is utilized.

Some implementations include one or more of the following features. The feedstock may include a plant that has recombinant DNA and/or recombinant genes. The modified plant may express one or more recombinant materials, for example, a protein, a polymer and/or a macromolecule. The method may further include obtaining from the feedstock materials such as pharmaceuticals, nutriceuticals, proteins, fats, vitamins, oils, fiber, minerals, sugars, carbohydrates and alcohols. The feedstock can include a crop residue e.g., corn cobs and/or corn stover, wheat straw, or the feedstock can be a genetically modified corn, wheat or soybean plant. The method may further include treating the feedstock with an organism and/or enzyme, in some cases producing a sugar e.g., in the form of a solution or suspension. Optionally, the sugar can be fermented. The physical treatment can include irradiation of the feedstock. In some implementations, the irradiated feedstock may be utilized as an edible material, e.g., as an animal feed. If desired, an enzyme such as a cellulase can be added to the edible material, e.g., to increase the nutrient value release.

Irradiating may in some cases be performed using one or more electron beam devices. In some cases, irradiating comprises applying a total dose of from about 5 Mrad to about 50 Mrad of radiation to the feedstock. Irradiation can sterilize the material prior to further processing and or storage prior to use. In preferred implementations, irradiating reduces the recalcitrance of the feedstock.

The plant may have been modified, for example, with a modification including enhancement of resistance to insects, fungal diseases, and other pests and disease-causing agents; increased tolerance to herbicides; increased drought resistance; extended temperature range; enhanced tolerance to poor soil; enhanced stability or shelf-life; greater yield; larger fruit size; stronger stalks; enhanced shatter resistance; reduced time to crop maturity; more uniform germination times; higher or modified starch production; enhanced nutrient production, such as enhanced, steroid, sterol, hormone, fatty acid, glycerol, polyhydroxyalkanoate, amino acid, vitamin and/or protein production; modified lignin content; enhanced cellulose, hemicellulose and/or lignin degradation; including of a phenotype marker to allow qualitative detection; reduced recalcitrance and enhanced phytate metabolism. The plant may be, for example, a genetically modified alfalfa, potato, beet, corn, wheat, cotton, rapeseed, rice, or sugarcane plant. The feedstock may include a crop residue from a modified plant, for example, the feedstock may include corn cobs and/or corn stover. The plant may be, for example, a genetically modified corn or soybean plant, or any of the many genetically modified plants that are grown.

In another aspect, the invention features a product comprising sugar derived from a feedstock obtained at least in part from a plant that has been modified with respect to a wild type variety of the plant, for example the plant has been genetically modified.

In a further aspect, the invention features a product comprising an irradiated cellulosic or lignocellulosic feedstock obtained at least in part from a plant that has been modified with respect to a wild type variety of the plant. The product may further include a microorganism and/or an enzyme, and in some cases a liquid medium.

Without being bound by any theory, it is believed that the use of modified plants can be advantageous over the non-modified wild type. For example, an enhancement of resistance to insects, fungal diseases, and other pests and disease-causing agents; an increased tolerance to herbicides; increased drought resistance; an extended temperature range; enhanced tolerance to poor soil; a larger fruit size; stronger stalks; enhanced shatter resistance; reduced time to crop maturity; more uniform germination times; can provide higher yields and a more varied feedstock source, both of which can lower the biomass feedstock cost. In another example, enhanced stability or shelf-life can be advantageous to biomass inventory quality. As another example, enhanced nutrient production, such as enhanced steroid, sterol, hormone, fatty acid, glycerol, polyhydroxyalkanoate, amino acid, vitamin and/or protein production can provide products or intermediates with higher nutrient quality that may improve a process e.g., a fermentation, or a product, e.g., an animal feed. Furthermore, for example, higher or modified starch production, modified lignin content; and/or enhanced cellulose, hemicellulose and/or lignin degradation can reduce the recalcitrance of the feedstock making it easier to process.

The term “plant,” as used herein, refers to any of various photosynthetic, eukaryotic, multicellular organisms of the kingdom Plantae, including but not limited to agricultural crops, trees, grasses, and algae.

“Structurally modifying” a feedstock, as that phrase is used herein, means changing the molecular structure of the feedstock in any way, including the chemical bonding arrangement, crystalline structure, or conformation of the feedstock. The change may be, for example, a change in the integrity of the crystalline structure, e.g., by microfracturing within the structure, which may not be reflected by diffractive measurements of the crystallinity of the material. Such changes in the structural integrity of the material can be measured indirectly by measuring the yield of a product at different levels of structure-modifying treatment. In addition, or alternatively, the change in the molecular structure can include changing the supramolecular structure of the material, oxidation of the material, changing an average molecular weight, changing an average crystallinity, changing a surface area, changing a degree of polymerization, changing a porosity, changing a degree of branching, grafting on other materials, changing a crystalline domain size, or changing an overall domain size.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents applications, patents and other references mentioned herein are incorporated by reference in their entirety. The materials, methods, and examples are illustrative only and not intended to be limiting.

Other features and advantages will be apparent from the following detailed description, and from the claims.

### DESCRIPTION OF DRAWINGS

FIG. 1 is a block diagram illustrating conversion of a feedstock into products and co-products.

FIG. 2 is a block diagram illustrating treatment of the feedstock and the use of the treated feedstock in a fermentation process.

### DETAILED DESCRIPTION

Feedstocks that are obtained from plants that have been modified with respect to a wild type variety, e.g., by genetic modification or other types of modification, can be processed to produce useful intermediates and products such as those described herein. Systems and processes are described herein that can use as feedstock materials e.g., cellulosic and/or lignocellulosic materials that are readily available, but can be difficult to process by processes such as fermentation. Many of the processes described herein can effectively lower the recalcitrance level of the feedstock, making it easier to process, such as by bioprocessing (e.g., with any microorganism described herein, such as a homoacetogen or a heteroacetogen, and/or any enzyme described herein), thermal processing (e.g., gasification or pyrolysis) or chemical methods (e.g., acid hydrolysis or oxidation). The feedstock can be treated or processed using one or more of any of the methods described herein, such as mechanical treatment, chemical treatment, radiation, sonication, oxidation, pyrolysis or steam explosion. The various treatment systems and methods can be used in combinations of two, three, or even four or more of these technologies or others described herein and elsewhere.

In addition to reducing the recalcitrance, the methods outlined above can also sterilize lignocellulosic or cellulosic feedstocks. This can be advantageous because feedstocks can be infected with, for example, a bacteria, a yeast, an insect and/or a fungus, that may have a deleterious effect on further processes and/or prematurely degrade the materials.

Feedstock materials, such as cellulosic and lignocellulosic feedstock materials, can be obtained from plants that have been modified with respect to a wild type variety. Such modifications may be for example, by any of the methods described in any patent or patent application referenced herein. As another example, plants may be modified through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogeneous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers.

Feedstock can be derived from a plant including, but not limited to canola, crambe, coconut, maize, mustard, castor bean, sesame, cottonseed, linseed, soybean, Arabidopsis phaseolus, peanut, alfalfa, wheat, rice, oat, sorghum, rapeseed, rye, tritordeum, millet, fescue, rye grass, sugarcane, cranberry, papaya, banana, safflower, oil palms, flax, muskmelon, apple, cucumber, dendrobium, gladiolus, chrysanthemum, liliaceae, cotton, eucalyptus, sunflower, Brassica campestris, Brassica napus, turfgrass, switch grass, cord grass, sugarbeet, coffee, dioscorea, acacia, apricot, artichoke, arugula, asparagus, avocado, barley, beans, beet, blackberry, blueberry, broccoli, brussels sprouts, cabbage, cantaloupe, carrot, cassava, cauliflower, celery, cherry, cilantro, clementine, corn, cotton, Douglas fir, bamboo, seaweed, algae, eggplant, endive, escarole, fennel, figs, forest tree, gourd, grape, grapefruit, honey dew, jicama, kiwifruit, lettuce, leeks, lemon, lime, loblolly pine, mango, melon, mushroom, nut, oat, okra, onion, orange, parsley, pea, peach, pear, pepper, persimmon, pine, pineapple, plantain, plum, pomegranate, poplar, potato, oryza sativa, pumpkin, quince, radiata pine, radicchio, radish, raspberry, rye, southern pine, soybean, spinach, squash, strawberry, sweet potato, sweetgum, tangerine, tea, tobacco, tomato, watermelon, wheat, yams, zucchini or mixtures of these. Preferably the feedstock material is derived from plant material not suitable for human consumption such as wood, agricultural waste, grasses such as switchgrass or miscanthus, rice hulls, bagasse, cotton, jute, hemp, flax, bamboo, sisal, abaci, straw, corn cobs, corn stover, hay, coconut hair, seaweed, algae or mixtures of these.

The advantages of plant modification include, for example, an enhancement of resistance to insects, fungal diseases, and other pests and disease-causing agents; an increased tolerance to herbicides; increased drought resistance; an extended temperature range; enhanced tolerance to poor soil; enhanced stability or shelf-life; a greater yield; larger fruit size; stronger stalks; enhanced shatter resistance; reduced time to crop maturity; more uniform germination times; higher or modified starch production; enhanced nutrient production, such as enhanced steroid, sterol, hormone, fatty acid, glycerol, polyhydroxyalkanoate, amino acid, vitamin and/or protein production; modified lignin content; enhanced cellulose, hemicellulose and/or lignin degradation; inclusion of a phenotype marker to allow qualitative detection (e.g., seed coat color); and modified phytate content. Any feedstock materials derived from these modified plants can also benefit from these many advantages. For example, a feedstock material such as a lignocellulosic material can have better shelf life, be easier to process, have a better land-to-energy conversion ratio, and/or have a better nutritional value to any microbes that are used in processing of the lignocellulosic material. In addition, any feedstock material derived from such plants can be less expensive and/or more plentiful. In some cases, modified plants can be grown in a greater variety of climates and/or soil types, for example, in marginal or depleted soils.

Feedstock materials can be obtained from modified plants having an increased resistance to disease. For example, potatoes which have reduced symptoms from the infestation of fungal pathogen Phytophthora infestans are discussed in U.S. Pat. No. 7,122,719. A possible advantage of such resistance is that the yield, quality and shelf life of the feedstock materials may be improved.

Feedstock materials can be obtained from modified plants with increased resistance to parasites, for example, by encoding genes for the production of 6-endotoxins as exemplified in U.S. Pat. No. 6,023,013. A possible advantage of such resistance is that the yield, quality and shelf life of the feedstock materials may be improved.

Feedstock materials can be obtained from modified plants having an increased resistance to herbicides. For example, the alfalfa plant J-101, as described in U.S. Pat. No. 7,566,817, has an increased resistance to glyphosphate herbicides. As a further example, modified plants described in U.S. Pat. No. 6,107,549 have an increased resistance to pyridine family herbicides. Furthermore, modified plants described in U.S. Pat. No. 7,498,429 have increased resistance to imidazolinones. A possible advantage of such resistance is that the yield and quality of the feedstock materials may be improved.

Feedstock materials can be obtained from modified plants having an increased stress resistance (for example, water deficit, cold, heat, salt, pest, disease, or nutrient stress). For example, such plants have been described in U.S. Pat. No. 7,674,952. A possible advantage of such resistance is that the yield and quality of the feedstock materials may be improved. Moreover, such plants may be grown in adverse conditions, e.g., marginal or depleted soil or in a harsh climate.

Feedstock materials can be obtained from modified plants with improved characteristics such as larger fruits. Such plants have been described in U.S. Pat. No. 7,335,812. A possible advantage of such resistance is that the yield and quality of the feedstock materials may be improved.

Feedstock materials can be obtained from modified plants with improved characteristics such as reduced pod shatter. Such plants have been described in U.S. Pat. No. 7,659,448. A possible advantage of such resistance is that the yield and quality of the feedstock materials may be improved.

Feedstock materials can be obtained from modified plants having enhanced or modified starch content. Such plants have been described in U.S. Pat. No. 6,538,178. A possible advantage of such modification is that the quality of the feedstock is improved.

Feedstock materials can be obtained from modified plants with a modified oil, fatty acid or glycol production. Such plants have been described in U.S. Pat. No. 7,405,344. Fatty acids and oils are excellent substrates for microbial energy-yielding metabolism and may provide an advantage to downstream processing of the feedstock for, for example, fuel production. Fatty acids and oil variation may also be advantageous in changing the viscosity and solubility of various components during downstream processing of the feedstock. The spent feedstock may have a better nutrient mix for use as animal feed or have higher calorie content useful as a direct fuel for burning.

Feedstock materials can be obtained from modified plants with a modified steroid, sterol and hormone content. Such plants have been described in U.S. Pat. No. 6,822,142. A possible advantage is that this may provide a better nutrient mix for microorganisms used in processing of the feedstock. After processing, the spent feedstock may have a better nutrient mix for use as animal feed.

Feedstock materials can be obtained from modified plants with polyhydroxyalkanoate producing ability. Such plants have been described in U.S. Pat. No. 6,175,061. Polyhydroxyalkanoates are a useful energy and carbon reserve for various microorganisms and may be beneficial to the microorganisms used in downstream feedstock processing. Also, since polyhydroxyalkanoate is biodegradable, it may impart advantages by possibly reducing recalcitrance in plant material after an aging period of the stored feedstock. Further downstream, the spent feedstock may have a better nutrient mix for use as animal feed or have higher calorie content useful as a direct fuel for burning.

Feedstock materials can be obtained from modified plants with enhanced amino acid production. Such plants have been described in U.S. Pat. No. 7,615,621. A possible advantage is that this may provide a better nutrient mix for microorganisms used in processing of the feedstock. After processing, the spent feedstock may have a better nutrient mix for use as animal feed.

Feedstock materials can be obtained from modified plants with elevated synthesis of vitamins. Such plants have been described in U.S. Pat. No. 6,841,717. A possible advantage is that this may provide a better nutrient mix for microorganisms used in processing of the feedstock. After processing, the spent feedstock may have a better nutrient mix for use as animal feed.

Feedstock materials can be obtained from modified plants that degrade lignin and cellulose in the plant after harvest. Such plants have been described in U.S. Pat. No. 7,049,485. Feedstock materials can also be obtained from modified plants with modified lignin content. Such plants have been described in U.S. Pat. No. 7,799,906. A possible advantage of such plants is reduced recalcitrance relative to the wild types of the same plants.

Feedstock materials can be obtained from modified plants with a modified phenotype for easy qualitative detection. Such plants have been described in U.S. Pat. No. 7,402,731. A possible advantage is ease of managing crops and seeds for different product streams such as biofuels, building materials and animal feed.

Feedstock materials can be obtained from modified plants with a reduced amount of phytate. Such plants have been described in U.S. Pat. No. 7,714,187. A possible advantage is that this may provide a better nutrient mix for microorganisms used in processing of the feedstock. After processing, the spent feedstock may have a better nutrient mix for use as animal feed.

Modified plants and/or plant materials and methods for making such modifications have been described in the U.S. Patents and U.S. Published applications listed at the end of this document (immediately before the claims), the entire disclosure of each of which is hereby incorporated by reference herein in its entirety.

### Systems for Treating a Feedstock

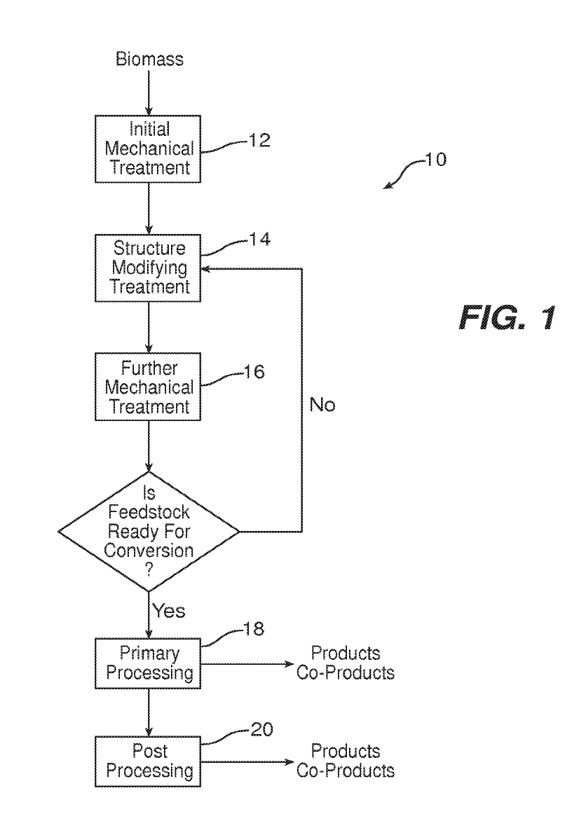

FIG. 1 shows one particular process for converting a feedstock, particularly a feedstock obtained at least in part from a modified plant material, into useful intermediates and products. Process 10 includes initially mechanically treating the feedstock (12), e.g., to reduce the size of the feedstock 110. The mechanically treated feedstock is then treated with a physical treatment (14) to modify its structure, for example, by weakening or microfracturing bonds in the crystalline structure of the material. Next, the structurally modified material may in some cases be subjected to further mechanical treatment (16). This mechanical treatment can be the same as or different from the initial mechanical treatment. For example, the initial treatment can be a size reduction (e.g., cutting) step followed by a shearing step, while the further treatment can be a grinding or milling step.

The material can then be subjected to further structure-modifying treatment and mechanical treatment, if further structural change (e.g., reduction in recalcitrance) is desired prior to further processing.

Next, the treated material can be processed with a primary processing step 18, e.g., saccharification and/or fermentation, to produce intermediates and products (e.g., energy, fuel, foods and materials). In some cases, the output of the primary processing step is directly useful but, in other cases, requires further processing provided by a post-processing step (20). For example, in the case of an alcohol, post-processing may involve distillation and, in some cases, denaturation.

As described herein, many variations of process 10 can be utilized.

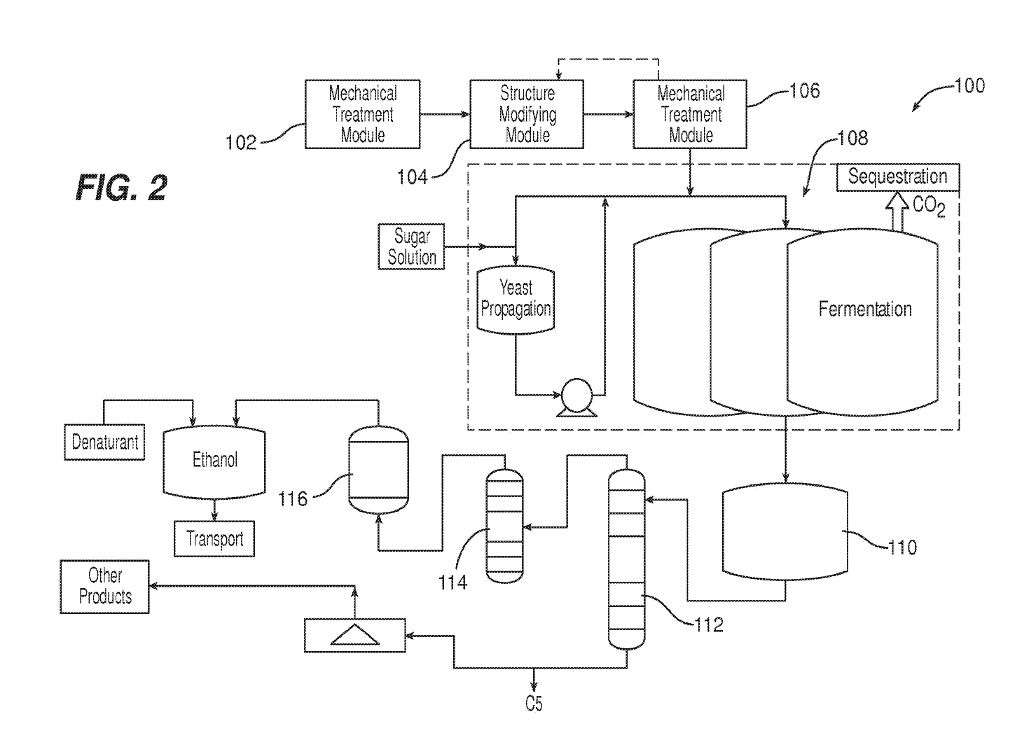

FIG. 2 shows one particular system that utilizes the steps described above for treating a feedstock and then using the treated feedstock in a fermentation process to produce an alcohol. System 100 includes a module 102 in which a feedstock is initially mechanically treated (step 12, above), a module 104 in which the mechanically treated feedstock is structurally modified (step 14, above), e.g., by irradiation, and a module 106 in which the structurally modified feedstock is subjected to further mechanical treatment (step 16, above). As discussed above, the module 106 may be of the same type as the module 102, or a different type. In some implementations the structurally modified feedstock can be returned to module 102 for further mechanical treatment rather than being further mechanically treated in a separate module 106.

As described herein, many variations of system 100 can be utilized.

After these treatments, which may be repeated as many times as required to obtain desired feedstock properties, the treated feedstock is delivered to a fermentation system 108. Mixing may be performed during fermentation, in which case the mixing is preferably relatively gentle (low shear) so as to minimize damage to shear sensitive ingredients such as enzymes and other microorganisms. In some embodiments, jet mixing is used, as described in U.S. Serial Nos. 12/782,694, 13/293,977 and 13/293,985, the complete disclosures of which are incorporated herein by reference.

Referring again to FIG. 2, fermentation produces a crude ethanol mixture, which flows into a holding tank 110. Water or other solvent, and other non-ethanol components, are stripped from the crude ethanol mixture using a stripping column 112, and the ethanol is then distilled using a distillation unit 114, e.g., a rectifier. Distillation may be by vacuum distillation. Finally, the ethanol can be dried using a molecular sieve 116 and/or denatured, if necessary, and output to a desired shipping method.

In some cases, the systems described herein, or components thereof, may be portable, so that the system can be transported (e.g., by rail, truck, or marine vessel) from one location to another. The method steps described herein can be performed at one or more locations, and in some cases one or more of the steps can be performed in transit. Such mobile processing is described in U.S. Ser. No. 12/374,549 and International Publication No. WO 2008/011598, the full disclosures of which are incorporated herein by reference.

Any or all of the method steps described herein can be performed at ambient temperature. If desired, cooling and/or heating may be employed during certain steps. For example, the feedstock may be cooled during mechanical treatment to increase its brittleness. In some embodiments, cooling is employed before, during or after the initial mechanical treatment and/or the subsequent mechanical treatment. Cooling may be performed as described in U.S. Ser. No. 12/502,629, now U.S. Pat. No. 7,900,857 the full disclosure of which is incorporated herein by reference. Moreover, the temperature in the fermentation system 108 may be controlled to enhance saccharification and/or fermentation.

The individual steps of the methods described above, as well as the materials used, will now be described in further detail.

### Physical Treatment

Physical treatment processes can include one or more of any of those described herein, such as mechanical treatment, chemical treatment, irradiation, sonication, oxidation, pyrolysis or steam explosion. Treatment methods can be used in combinations of two, three, four, or even all of these technologies (in any order). When more than one treatment method is used, the methods can be applied at the same time or at different times. Other processes that change a molecular structure of a feedstock may also be used, alone or in combination with the processes disclosed herein.

### Mechanical Treatments

In some cases, methods can include mechanically treating the feedstock. Mechanical treatments include, for example, cutting, milling, pressing, grinding, shearing and chopping. Milling may include, for example, ball milling, hammer milling, rotor/stator dry or wet milling, freezer milling, blade milling, knife milling, disk milling, roller milling or other types of milling. Other mechanical treatments include, e.g., stone grinding, cracking, mechanical ripping or tearing, pin grinding or air attrition milling.

Mechanical treatment can be advantageous for “opening up,” “stressing,” breaking and shattering cellulosic or lignocellulosic materials in the feedstock, making the cellulose of the materials more susceptible to chain scission and/or reduction of crystallinity. The open materials can also be more susceptible to oxidation when irradiated.

In some cases, the mechanical treatment may include an initial preparation of the feedstock as received, e.g., size reduction of materials, such as by cutting, grinding, shearing, pulverizing or chopping. For example, in some cases, loose feedstock (e.g., recycled paper, starchy materials, or switchgrass) is prepared by shearing or shredding.

Alternatively, or in addition, the feedstock material can first be physically treated by one or more of the other physical treatment methods, e.g., chemical treatment, radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the molecular structure of the material by mechanical treatment.

In some embodiments, the feedstock is in the form of a fibrous material, and mechanical treatment includes shearing to expose fibers of the fibrous material. Shearing can be performed, for example, using a rotary knife cutter. Other methods of mechanically treating the feedstock include, for example, milling or grinding. Milling may be performed using, for example, a hammer mill, ball mill, colloid mill, conical or cone mill, disk mill, edge mill, Wiley mill or grist mill. Grinding may be performed using, for example, a stone grinder, pin grinder, coffee grinder, or burr grinder. Grinding may be provided, for example, by a reciprocating pin or other element, as is the case in a pin mill. Other mechanical treatment methods include mechanical ripping or tearing, other methods that apply pressure to the material, and air attrition milling. Suitable mechanical treatments further include any other technique that changes the molecular structure of the feedstock.

If desired, the mechanically treated material can be passed through a screen, e.g., having an average opening size of 1.59 mm or less ( 1/16 inch, 0.0625 inch). In some embodiments, shearing, or other mechanical treatment, and screening are performed concurrently. For example, a rotary knife cutter can be used to concurrently shear and screen the feedstock. The feedstock is sheared between stationary blades and rotating blades to provide a sheared material that passes through a screen, and is captured in a bin.

The feedstock can be mechanically treated in a dry state (e.g., having little or no free water on its surface), a hydrated state (e.g., having up to ten percent by weight absorbed water), or in a wet state, e.g., having between about 10 percent and about 75 percent by weight water. The fiber source can even be mechanically treated while partially or fully submerged under a liquid, such as water, ethanol or isopropanol.

The feedstock can also be mechanically treated under a gas (such as a stream or atmosphere of gas other than air), e.g., oxygen or nitrogen, or steam.

If desired, lignin can be removed from any of the fibrous materials that include lignin. Also, to aid in the breakdown of the materials that include cellulose, the material can be treated prior to or during mechanical treatment or irradiation with heat, a chemical (e.g., mineral acid, base or a strong oxidizer such as sodium hypochlorite) and/or an enzyme. For example, grinding can be performed in the presence of an acid.

Mechanical treatment systems can be configured to produce streams with specific morphology characteristics such as, for example, surface area, porosity, bulk density, and, in the case of fibrous feedstocks, fiber characteristics such as length-to-width ratio.

In some embodiments, a BET surface area of the mechanically treated material is greater than 0.1 m²/g, e.g., greater than 0.25 m²/g, greater than 0.5 m²/g, greater than 1.0 m²/g, greater than 1.5 m²/g, greater than 1.75 m²/g, greater than 5.0 m²/g, greater than 10 m²/g, greater than 25 m²/g, greater than 35 m²/g, greater than 50 m²/g, greater than 60 m²/g, greater than 75 m²/g, greater than 100 m²/g, greater than 150 m²/g, greater than 200 m²/g, or even greater than 250 m²/g.

A porosity of the mechanically treated material can be, e.g., greater than 20 percent, greater than 25 percent, greater than 35 percent, greater than 50 percent, greater than 60 percent, greater than 70 percent, greater than 80 percent, greater than 85 percent, greater than 90 percent, greater than 92 percent, greater than 94 percent, greater than 95 percent, greater than 97.5 percent, greater than 99 percent, or even greater than 99.5 percent.

In some embodiments, after mechanical treatment the material has a bulk density of less than 0.75 g/cm³, e.g., less than about 0.7, 0.65, 0.60, 0.50, 0.35, 0.25, 0.20, 0.15, 0.10, 0.05, or less, e.g., less than 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters.

If the feedstock is a fibrous material the fibers of the mechanically treated material can have a relatively large average length-to-diameter ratio (e.g., greater than 20-to-1), even if they have been sheared more than once. In addition, the fibers of the fibrous materials described herein may have a relatively narrow length and/or length-to-diameter ratio distribution.

As used herein, average fiber widths (e.g., diameters) are those determined optically by randomly selecting approximately 5,000 fibers. Average fiber lengths are corrected length-weighted lengths. BET (Brunauer, Emmet and Teller) surface areas are multi-point surface areas, and porosities are those determined by mercury porosimetry.

If the feedstock is a fibrous material the average length-to-diameter ratio of fibers of the mechanically treated material can be, e.g., greater than 8/1, e.g., greater than 10/1, greater than 15/1, greater than 20/1, greater than 25/1, or greater than 50/1. An average fiber length of the mechanically treated material can be, e.g., between about 0.5 mm and 2.5 mm, e.g., between about 0.75 mm and 1.0 mm, and an average width (e.g., diameter) of the second fibrous material 14 can be, e.g., between about 5 μm and 50 μm, e.g., between about 10 μm and 30 μm.

In some embodiments, if the feedstock is a fibrous material, the standard deviation of the fiber length of the mechanically treated material can be less than 60 percent of an average fiber length of the mechanically treated material, e.g., less than 50 percent of the average length, less than 40 percent of the average length, less than 25 percent of the average length, less than 10 percent of the average length, less than 5 percent of the average length, or even less than 1 percent of the average length.

In some situations, it can be desirable to prepare a low bulk density material, densify the material (e.g., to make it easier and less costly to transport to another site), and then revert the material to a lower bulk density state. Densified materials can be processed by any of the methods described herein, or any material processed by any of the methods described herein can be subsequently densified, e.g., as disclosed in U.S. Ser. No. 12/429,045 now U.S. Pat. No. 7,932,065 and WO 2008/073186, the full disclosures of which are incorporated herein by reference.

### Radiation Treatment

One or more radiation processing sequences can be used to process the feedstock, and to provide a structurally modified material which functions as input to further processing steps and/or sequences. Irradiation can, for example, reduce the molecular weight and/or crystallinity of feedstock. Radiation can also sterilize the materials, or any media needed to bioprocess the material.

In some embodiments, energy deposited in a material that releases an electron from its atomic orbital is used to irradiate the materials. The radiation may be provided by (1) heavy charged particles, such as alpha particles or protons, (2) electrons, produced, for example, in beta decay or electron beam accelerators, or (3) electromagnetic radiation, for example, gamma rays, x rays, or ultraviolet rays. In one approach, radiation produced by radioactive substances can be used to irradiate the feedstock. In another approach, electromagnetic radiation (e.g., produced using electron beam emitters) can be used to irradiate the feedstock. In some embodiments, any combination in any order or concurrently of (1) through (3) may be utilized. The doses applied depend on the desired effect and the particular feedstock.

In some instances when chain scission is desirable and/or polymer chain functionalization is desirable, particles heavier than electrons, such as protons, helium nuclei, argon ions, silicon ions, neon ions, carbon ions, phosphorus ions, oxygen ions or nitrogen ions can be utilized. When ring-opening chain scission is desired, positively charged particles can be utilized for their Lewis acid properties for enhanced ring-opening chain scission. For example, when maximum oxidation is desired, oxygen ions can be utilized, and when maximum nitration is desired, nitrogen ions can be utilized. The use of heavy particles and positively charged particles is described in U.S. Ser. No. 12/417,699, now U.S. Pat. No. 7,931,784, the full disclosure of which is incorporated herein by reference.

In one method, a first material that is or includes cellulose having a first number average molecular weight (M_{N1}) is irradiated, e.g., by treatment with ionizing radiation (e.g., in the form of gamma radiation, X-ray radiation, 100 nm to 280 nm ultraviolet (UV) light, a beam of electrons or other charged particles) to provide a second material that includes cellulose having a second number average molecular weight (M_{N2}) lower than the first number average molecular weight. The second material (or the first and second material) can be combined with a microorganism (with or without enzyme treatment) that can utilize the second and/or first material or its constituent sugars or lignin to produce an intermediate or product, such as those described herein.

Since the second material includes cellulose having a reduced molecular weight relative to the first material, and in some instances, a reduced crystallinity as well, the second material is generally more dispersible, swellable and/or soluble, e.g., in a solution containing a microorganism and/or an enzyme. These properties make the second material easier to process and more susceptible to chemical, enzymatic and/or biological attack relative to the first material, which can greatly improve the production rate and/or production level of a desired product, e.g., ethanol.

In some embodiments, the second number average molecular weight (M_{N2}) is lower than the first number average molecular weight (M_{N1}) by more than about 10 percent, e.g., more than about 15, 20, 25, 30, 35, 40, 50 percent, 60 percent, or even more than about 75 percent.

In some instances, the second material includes cellulose that has a crystallinity (C₂) that is lower than the crystallinity (C₁) of the cellulose of the first material. For example, (C₂) can be lower than (C₁) by more than about 10 percent. e.g., more than about 15, 20, 25, 30, 35, 40, or even more than about 50 percent.

In some embodiments, the starting crystallinity index (prior to irradiation) is from about 40 to about 87.5 percent, e.g., from about 50 to about 75 percent or from about 60 to about 70 percent, and the crystallinity index after irradiation is from about 10 to about 50 percent, e.g., from about 15 to about 45 percent or from about 20 to about 40 percent. However, in some embodiments, e.g., after extensive irradiation, it is possible to have a crystallinity index of lower than 5 percent. In some embodiments, the material after irradiation is substantially amorphous.

In some embodiments, the starting number average molecular weight (prior to irradiation) is from about 200,000 to about 3,200,000, e.g., from about 250,000 to about 1,000,000 or from about 250,000 to about 700,000, and the number average molecular weight after irradiation is from about 50,000 to about 200,000, e.g., from about 60,000 to about 150,000 or from about 70,000 to about 125,000. However, in some embodiments, e.g., after extensive irradiation, it is possible to have a number average molecular weight of less than about 10,000 or even less than about 5,000.

In some embodiments, the second material can have a level of oxidation (O₂) that is higher than the level of oxidation (O₁) of the first material. A higher level of oxidation of the material can aid in its dispersability, swellability and/or solubility, further enhancing the material's susceptibility to chemical, enzymatic or biological attack. In some embodiments, to increase the level of the oxidation of the second material relative to the first material, the irradiation is performed under an oxidizing environment, e.g., under a blanket of air or oxygen, producing a second material that is more oxidized than the first material. For example, the second material can have more hydroxyl groups, aldehyde groups, ketone groups, ester groups or carboxylic acid groups, which can increase its hydrophilicity.

Ionizing Radiation

Each form of radiation ionizes the carbon-containing material via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired, positively charged particles may be desirable, in part due to their acidic nature. When particles are utilized, the particles can have the mass of a resting electron, or greater, e.g., 500, 1000, 1500, 2000, 10,000 or even 100,000 times the mass of a resting electron. For example, the particles can have a mass of from about 1 atomic unit to about 150 atomic units, e.g., from about 1 atomic unit to about 50 atomic units, or from about 1 to about 25. e.g., 1, 2, 3, 4, 5, 10, 12 or 15 amu. Accelerators used to accelerate the particles can be electrostatic DC, electrodynamic DC, RF linear, magnetic induction linear or continuous wave. For example, cyclotron type accelerators are available from IBA, Belgium, such as the RHODOTRON® system, while DC type accelerators are available from RDI, now IBA Industrial, such as the DYNAMITRON®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy” Columbus-Ohio, ICRU-IAEA Meeting, 18-20 Mar. 2006. Iwata, Y. et al., “Alternating-Phase-Focused IH-DTL for Heavy-Ion Medical Accelerators” Proceedings of EPAC 2006, Edinburgh, Scotland and Leaner, C. M. et al., “Status of the Superconducting ECR Ion Source Venus” Proceedings of EPAC 2000, Vienna, Austria.

Gamma radiation has the advantage of a significant penetration depth into a variety of materials. Sources of gamma rays include radioactive nuclei, such as isotopes of cobalt, calcium, technicium, chromium, gallium, indium, iodine, iron, krypton, samarium, selenium, sodium, thalium, and xenon.

Sources of x rays include electron beam collision with metal targets, such as tungsten or molybdenum or alloys, or compact light sources, such as those produced commercially by Lyncean.

Sources for ultraviolet radiation include deuterium or cadmium lamps.

Sources for infrared radiation include sapphire, zinc, or selenide window ceramic lamps.

Sources for microwaves include klystrons, Slevin type RF sources, or atom beam sources that employ hydrogen, oxygen, or nitrogen gases.

In some embodiments, a beam of electrons is used as the radiation source. A beam of electrons has the advantages of high dose rates (e.g., 1, 5, or even 10 Mrad per second), high throughput, less containment, and less confinement equipment. Electrons can also be more efficient at causing chain scission. In addition, electrons having energies of 4-10 MeV can have a penetration depth of 5 to 30 mm or more, such as 40 mm.

Electron beams can be generated, e.g., by electrostatic generators, cascade generators, transformer generators, low energy accelerators with a scanning system, low energy accelerators with a linear cathode, linear accelerators, and pulsed accelerators. Electrons as an ionizing radiation source can be useful, e.g., for relatively thin sections of material, e.g., less than 0.5 inch, e.g., less than 0.4 inch, 0.3 inch, 0.2 inch, or less than 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV.

Electron beam irradiation devices may be procured commercially from Ion Beam Applications, Louvain-la-Neuve, Belgium or the Titan Corporation, San Diego, Calif. Typical electron energies can be 1 MeV, 2 MeV, 4.5 MeV, 7.5 MeV, or 10 MeV. Typical electron beam irradiation device power can be 1 kW, 5 kW, 10 kW, 20 kW, 50 kW, 100 kW, 250 kW, or 500 kW. The level of depolymerization of the feedstock depends on the electron energy used and the dose applied, while exposure time depends on the power and dose. Typical doses may take values of 1 kGy, 5 kGy, 10 kGy, 20 kGy, 50 kGy, 100 kGy, or 200 kGy. In some embodiments, energies between 0.25-10 MeV (e.g., 0.5-0.8 MeV, 0.5-5 MeV, 0.8-4 MeV, 0.8-3 MeV, 0.8-2 MeV or 0.8-1.5 MeV) can be used. In some embodiments, doses between 1-100 Mrad (e.g., 2-80 Mrad, 5-50 Mrad, 5-40 Mrad, 5-30 Mrad or 5-20 Mrad) can be used. In some preferred embodiments, an energy between 0.8-3 MeV (e.g., 0.8-2 MeV or 0.8-1.5 MeV) combined with doses between 5-50 Mrad (e.g., 5-40 Mrad, 5-30 Mrad or 5-20 Mrad) can be used.

Ion Particle Beams

Particles heavier than electrons can be utilized to irradiate materials, such as carbohydrates or materials that include carbohydrates, e.g., cellulosic materials, lignocellulosic materials, starchy materials, or mixtures of any of these and others described herein. For example, protons, helium nuclei, argon ions, silicon ions, neon ions carbon ions, phosphorus ions, oxygen ions or nitrogen ions can be utilized. In some embodiments, particles heavier than electrons can induce higher amounts of chain scission (relative to lighter particles). In some instances, positively charged particles can induce higher amounts of chain scission than negatively charged particles due to their acidity.

Heavier particle beams can be generated, e.g., using linear accelerators or cyclotrons. In some embodiments, the energy of each particle of the beam is from about 1.0 MeV/atomic unit (MeV/amu) to about 6,000 MeV/atomic unit, e.g., from about 3 MeV/atomic unit to about 4,800 MeV/atomic unit, or from about 10 MeV/atomic unit to about 1,000 MeV/atomic unit.

In certain embodiments, ion beams used to irradiate carbon-containing materials, e.g., materials obtained from plants, can include more than one type of ion. For example, ion beams can include mixtures of two or more (e.g., three, four or more) different types of ions. Exemplary mixtures can include carbon ions and protons, carbon ions and oxygen ions, nitrogen ions and protons, and iron ions and protons. More generally, mixtures of any of the ions discussed above (or any other ions) can be used to form irradiating ion beams. In particular, mixtures of relatively light and relatively heavier ions can be used in a single ion beam.

In some embodiments, ion beams for irradiating materials include positively-charged ions. The positively charged ions can include, for example, positively charged hydrogen ions (e.g., protons), noble gas ions (e.g., helium, neon, argon), carbon ions, nitrogen ions, oxygen ions, silicon atoms, phosphorus ions, and metal ions such as sodium ions, calcium ions, and/or iron ions. Without wishing to be bound by any theory, it is believed that such positively-charged ions behave chemically as Lewis acid moieties when exposed to materials, initiating and sustaining cationic ring-opening chain scission reactions in an oxidative environment.

In certain embodiments, ion beams for irradiating materials include negatively-charged ions. Negatively charged ions can include, for example, negatively charged hydrogen ions (e.g., hydride ions), and negatively charged ions of various relatively electronegative nuclei (e.g., oxygen ions, nitrogen ions, carbon ions, silicon ions, and phosphorus ions). Without wishing to be bound by any theory, it is believed that such negatively-charged ions behave chemically as Lewis base moieties when exposed to materials, causing anionic ring-opening chain scission reactions in a reducing environment.

In some embodiments, beams for irradiating materials can include neutral atoms. For example, any one or more of hydrogen atoms, helium atoms, carbon atoms, nitrogen atoms, oxygen atoms, neon atoms, silicon atoms, phosphorus atoms, argon atoms, and iron atoms can be included in beams that are used for irradiation. In general, mixtures of any two or more of the above types of atoms (e.g., three or more, four or more, or even more) can be present in the beams.

In certain embodiments, ion beams used to irradiate materials include singly-charged ions such as one or more of H⁺, H⁻, He⁺, Ne⁺, Ar⁺, C⁺, C⁻, O⁺, O⁻, N⁺, N⁻, Si⁺, Si⁻, P⁺, P⁻, Na⁺, Ca⁺, and Fe⁺. In some embodiments, ion beams can include multiply-charged ions such as one or more of C²⁺, C³⁺, C⁴⁺, N³⁺, N⁵⁺, N³⁻, O²⁺, O²⁻, O₂²⁻, Si²⁺, Si⁴⁺, Si²⁻, and Si⁴⁻. In general, the ion beams can also include more complex polynuclear ions that bear multiple positive or negative charges. In certain embodiments, by virtue of the structure 15s of the polynuclear ion, the positive or negative charges can be effectively distributed over substantially the entire structure of the ions. In some embodiments, the positive or negative charges can be somewhat localized over portions of the structure of the ions.

Electromagnetic Radiation

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ hz, greater than 10¹⁷ hz, 10¹⁸, 10¹⁹, 10²⁰, or even greater than 10²1 hz. Typical doses may take values of greater than 1 Mrad (e.g., greater than 1 Mrad, greater than 2 Mrad). In some embodiments, the electromagnetic radiation has a frequency of between 10¹⁸ and 10²² hz, e.g., between 10¹⁹ to 10²¹ Hz. In some embodiment doses between 1-100 Mrad (e.g., 2-80 Mrad, 5-50 Mrad, 5-40 Mrad, 5-30 Mrad or 5-20 Mrad) can be used.

Quenching and Controlled Functionalization

After treatment with ionizing radiation, any of the materials or mixtures described herein may become ionized; that is, the treated material may include radicals at levels that are detectable with an electron spin resonance spectrometer. If an ionized feedstock remains in the atmosphere, it will be oxidized, such as to an extent that carboxylic acid groups are generated by reacting with the atmospheric oxygen. In some instances with some materials, such oxidation is desired because it can aid in the further breakdown in molecular weight of the carbohydrate-containing biomass, and the oxidation groups, e.g., carboxylic acid groups can be helpful for solubility and microorganism utilization in some instances. However, since the radicals can “live” for some time after irradiation, e.g., longer than 1 day, 5 days, 30 days, 3 months, 6 months or even longer than 1 year, material properties can continue to change over time, which in some instances, can be undesirable. Thus, it may be desirable to quench the ionized material.

After ionization, any ionized material can be quenched to reduce the level of radicals in the ionized material, e.g., such that the radicals are no longer detectable with the electron spin resonance spectrometer. For example, the radicals can be quenched by the application of a sufficient pressure to the material and/or by utilizing a fluid in contact with the ionized material, such as a gas or liquid, that reacts with (quenches) the radicals. Using a gas or liquid to at least aid in the quenching of the radicals can be used to functionalize the ionized material with a desired amount and kind of functional groups, such as carboxylic acid groups, enol groups, aldehyde groups, nitro groups, nitrile groups, amino groups, alkyl amino groups, alkyl groups, chloroalkyl groups or chlorofluoroalkyl groups.

In some instances, such quenching can improve the stability of some of the ionized materials. For example, quenching can improve the resistance of the material to oxidation. Functionalization by quenching can also improve the solubility of any material described herein, can improve its thermal stability, and can improve material utilization by various microorganisms. For example, the functional groups imparted to the material by the quenching can act as receptor sites for attachment by microorganisms, e.g., to enhance cellulose hydrolysis by various microorganisms.

In some embodiments, quenching includes an application of pressure to the ionized material, such as by mechanically deforming the material, e.g., directly mechanically compressing the material in one, two, or three dimensions, or applying pressure to a fluid in which the material is immersed, e.g., isostatic pressing. In such instances, the deformation of the material itself brings radicals, which are often trapped in crystalline domains, in close enough proximity so that the radicals can recombine, or react with another group. In some instances, the pressure is applied together with the application of heat, such as a sufficient quantity of heat to elevate the temperature of the material to above a melting point or softening point of a component of the material, such as lignin, cellulose or hemicellulose. Heat can improve molecular mobility in the material, which can aid in the quenching of the radicals. When pressure is utilized to quench, the pressure can be greater than about 1000 psi, such as greater than about 1250 psi, 1450 psi, 3625 psi, 5075 psi, 7250 psi, 10000 psi or even greater than 15000 psi.

In some embodiments, quenching includes contacting the ionized material with a fluid, such as a liquid or gas, e.g., a gas capable of reacting with the radicals, such as acetylene or a mixture of acetylene in nitrogen, ethylene, chlorinated ethylenes or chlorofluoroethylenes, propylene or mixtures of these gases. In other particular embodiments, quenching includes contacting the ionized material with a liquid, e.g., a liquid soluble in, or at least capable of penetrating into the material and reacting with the radicals, such as a diene, such as 1,5-cyclooctadiene. In some specific embodiments, quenching includes contacting the material with an antioxidant, such as Vitamin E. If desired, the feedstock can include an antioxidant dispersed therein, and the quenching can come from contacting the antioxidant dispersed in the feedstock with the radicals.

Functionalization can be enhanced by utilizing heavy charged ions, such as any of the heavier ions described herein. For example, if it is desired to enhance oxidation, charged oxygen ions can be utilized for the irradiation. If nitrogen functional groups are desired, nitrogen ions or anions that include nitrogen can be utilized. Likewise, if sulfur or phosphorus groups are desired, sulfur or phosphorus ions can be used in the irradiation.

Doses

In some instances, the irradiation is performed at a dosage rate of greater than about 0.25 Mrad per second, e.g., greater than about 0.5, 0.75, 1.0, 1.5, 2.0, or even greater than about 2.5 Mrad per second. In some embodiments, the irradiating is performed at a dose rate of between 5.0 and 1500.0 kilorads/hour, e.g., between 10.0 and 750.0 kilorads/hour or between 50.0 and 350.0 kilorads/hour. In some embodiments, irradiation is performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 2 Mrad per second.

In some embodiments, the irradiating (with any radiation source or a combination of sources) is performed until the material receives a dose of 0.25 Mrad, e.g., at least 1.0, 2.5, 5.0, 8.0, 10, 15, 20, 25, 30, 35, 40, 50, or even at least 100 Mrad. In some embodiments, the irradiating is performed until the material receives a dose of between 1.0 Mrad and 6.0 Mrad, e.g., between 1.5 Mrad and 4.0 Mrad, 2 Mrad and 10 Mrad, 5 Mrad and 20 Mrad, 10 Mrad and 30 Mrad, 10 Mrad and 40 Mrad, or 20 Mrad and 50 Mrad. In some embodiments, the irradiating is performed until the material receives a dose of from about 0.1 Mrad to about 500 Mrad, from about 0.5 Mrad to about 200 Mrad, from about 1 Mrad to about 100 Mrad, or from about 5 Mrad to about 60 Mrad. In some embodiments, a relatively low dose of radiation is applied, e.g., less than 60 Mrad.

### Sonication

Sonication can reduce the molecular weight and/or crystallinity of materials, such as one or more of any of the materials described herein, e.g., one or more carbohydrate sources, such as cellulosic or lignocellulosic materials, or starchy materials. Sonication can also be used to sterilize the materials. As discussed above with regard to radiation, the process parameters used for sonication can be varied depending on various factors, e.g., depending on the lignin content of the feedstock. For example, feedstocks with higher lignin levels generally require a higher residence time and/or energy level, resulting in a higher total energy delivered to the feedstock.

In one method, a first material that includes cellulose having a first number average molecular weight (M_{N1}) is dispersed in a medium, such as water, and sonicated and/or otherwise cavitated, to provide a second material that includes cellulose having a second number average molecular weight (M_{N2}) lower than the first number average molecular weight. The second material (or the first and second material in certain embodiments) can be combined with a microorganism (with or without enzyme treatment) that can utilize the second and/or first material to produce an intermediate or product.

Since the second material includes cellulose having a reduced molecular weight relative to the first material, and in some instances, a reduced crystallinity as well, the second material is generally more dispersible, swellable, and/or soluble. e.g., in a solution containing a microorganism.

In some embodiments, the second number average molecular weight (M_{N2}) is lower than the first number average molecular weight (M_{N1}) by more than about 10 percent, e.g., more than about 15, 20, 25, 30, 35, 40, 50 percent, 60 percent, or even more than about 75 percent.

In some instances, the second material includes cellulose that has a crystallinity (C₂) that is lower than the crystallinity (C₁) of the cellulose of the first material. For example, (C₂) can be lower than (C₁) by more than about 10 percent, e.g., more than about 15, 20, 25, 30, 35, 40, or even more than about 50 percent.

In some embodiments, the starting crystallinity index (prior to sonication) is from about 40 to about 87.5 percent, e.g., from about 50 to about 75 percent or from about 60 to about 70 percent, and the crystallinity index after sonication is from about 10 to about 50 percent, e.g., from about 15 to about 45 percent or from about 20 to about 40 percent. However, in certain embodiments, e.g., after extensive sonication, it is possible to have a crystallinity index of lower than 5 percent. In some embodiments, the material after sonication is substantially amorphous.

In some embodiments, the starting number average molecular weight (prior to sonication) is from about 200,000 to about 3,200,000, e.g., from about 250,000 to about 1,000,000 or from about 250,000 to about 700,000, and the number average molecular weight after sonication is from about 50,000 to about 200,000, e.g., from about 60,000 to about 150,000 or from about 70,000 to about 125,000. However, in some embodiments, e.g., after extensive sonication, it is possible to have a number average molecular weight of less than about 10,000 or even less than about 5,000.

In some embodiments, the second material can have a level of oxidation (O₂) that is higher than the level of oxidation (O₁) of the first material. A higher level of oxidation of the material can aid in its dispersability, swellability and/or solubility, further enhancing the material's susceptibility to chemical, enzymatic or microbial attack. In some embodiments, to increase the level of the oxidation of the second material relative to the first material, the sonication is performed in an oxidizing medium, producing a second material that is more oxidized than the first material. For example, the second material can have more hydroxyl groups, aldehyde groups, ketone groups, ester groups or carboxylic acid groups, which can increase its hydrophilicity.

In some embodiments, the sonication medium is an aqueous medium. If desired, the medium can include an oxidant, such as a peroxide (e.g., hydrogen peroxide), a dispersing agent and/or a buffer. Examples of dispersing agents include ionic dispersing agents, e.g., sodium lauryl sulfate, and non-ionic dispersing agents, e.g., poly(ethylene glycol).

In other embodiments, the sonication medium is non-aqueous. For example, the sonication can be performed in a hydrocarbon, e.g., toluene or heptane, an ether, e.g., diethyl ether or tetrahydrofuran, or even in a liquefied gas such as argon, xenon, or nitrogen.

### Pyrolysis

One or more pyrolysis processing sequences can be used to process carbon-containing materials from a wide variety of different sources to extract useful substances from the materials, and to provide partially degraded materials which function as input to further processing steps and/or sequences. Pyrolysis can also be used to sterilize the materials. Pyrolysis conditions can be varied depending on the characteristics of the feedstock and/or other factors. For example, feedstocks with higher lignin levels may require a higher temperature, longer residence time, and/or introduction of higher levels of oxygen during pyrolysis.

In one example, a first material that includes cellulose having a first number average molecular weight (M_{N1}) is pyrolyzed, e.g., by heating the first material in a tube furnace (in the presence or absence of oxygen), to provide a second material that includes cellulose having a second number average molecular weight (M_{N2}) lower than the first number average molecular weight.

Since the second material includes cellulose having a reduced molecular weight relative to the first material, and in some instances, a reduced crystallinity as well, the second material is generally more dispersible, swellable and/or soluble, e.g., in a solution containing a microorganism.

In some embodiments, the second number average molecular weight (M_{N2}) is lower than the first number average molecular weight (M_{N1}) by more than about 10 percent, e.g., more than about 15, 20, 25, 30, 35, 40, 50 percent, 60 percent, or even more than about 75 percent.

In some instances, the second material includes cellulose that has a crystallinity (C₂) that is lower than the crystallinity (C₁) of the cellulose of the first material. For example, (C₂) can be lower than (C₁) by more than about 10 percent, e.g., more than about 15, 20, 25, 30, 35, 40, or even more than about 50 percent.

In some embodiments, the starting crystallinity (prior to pyrolysis) is from about 40 to about 87.5 percent, e.g., from about 50 to about 75 percent or from about 60 to about 70 percent, and the crystallinity index after pyrolysis is from about 10 to about 50 percent, e.g., from about 15 to about 45 percent or from about 20 to about 40 percent. However, in certain embodiments, e.g., after extensive pyrolysis, it is possible to have a crystallinity index of lower than 5 percent. In some embodiments, the material after pyrolysis is substantially amorphous.

In some embodiments, the starting number average molecular weight (prior to pyrolysis) is from about 200,000 to about 3,200,000. e.g., from about 250,000 to about 1,000,000 or from about 250,000 to about 700,000, and the number average molecular weight after pyrolysis is from about 50,000 to about 200,000, e.g., from about 60,000 to about 150,000 or from about 70,000 to about 125,000. However, in some embodiments, e.g., after extensive pyrolysis, it is possible to have a number average molecular weight of less than about 10,000 or even less than about 5,000.

In some embodiments, the second material can have a level of oxidation (O₂) that is higher than the level of oxidation (O₁) of the first material. A higher level of oxidation of the material can aid in its dispersability, swellability and/or solubility, further enhancing the susceptibility of the material to chemical, enzymatic or microbial attack. In some embodiments, to increase the level of the oxidation of the second material relative to the first material, the pyrolysis is performed in an oxidizing environment, producing a second material that is more oxidized than the first material. For example, the second material can have more hydroxyl groups, aldehyde groups, ketone groups, ester groups or carboxylic acid groups, than the first material, thereby increasing the hydrophilicity of the material.

In some embodiments, the pyrolysis of the materials is continuous. In other embodiments, the material is pyrolyzed for a pre-determined time, and then allowed to cool for a second pre-determined time before pyrolyzing again.

### Oxidation

One or more oxidative processing sequences can be used to process carbon-containing materials from a wide variety of different sources to extract useful substances from the materials, and to provide partially degraded and/or altered material which functions as input to further processing steps and/or sequences. The oxidation conditions can be varied, e.g., depending on the lignin content of the feedstock, with a higher degree of oxidation generally being desired for higher lignin content feedstocks.

In one method, a first material that includes cellulose having a first number average molecular weight (M_{N1}) and having a first oxygen content (O₁) is oxidized, e.g., by heating the first material in a stream of air or oxygen-enriched air, to provide a second material that includes cellulose having a second number average molecular weight (M_{N2}) and having a second oxygen content (O₂) higher than the first oxygen content (Os).

The second number average molecular weight of the second material is generally lower than the first number average molecular weight of the first material. For example, the molecular weight may be reduced to the same extent as discussed above with respect to the other physical treatments. The crystallinity of the second material may also be reduced to the same extent as discussed above with respect to the other physical treatments.

In some embodiments, the second oxygen content is at least about five percent higher than the first oxygen content, e.g., 7.5 percent higher, 10.0 percent higher, 12.5 percent higher, 15.0 percent higher or 17.5 percent higher. In some preferred embodiments, the second oxygen content is at least about 20.0 percent higher than the first oxygen content of the first material. Oxygen content is measured by elemental analysis by pyrolyzing a sample in a furnace operating at 1300° C. or higher. A suitable CHN elemental analyzer is the LECO® CHNS-932 analyzer with a VTF-900 high temperature pyrolysis furnace.

Generally, oxidation of a material occurs in an oxidizing environment. For example, the oxidation can be effected or aided by pyrolysis in an oxidizing environment, such as in air or argon enriched in air. To aid in the oxidation, various chemical agents, such as oxidants, acids or bases can be added to the material prior to or during oxidation. For example, a peroxide (e.g., benzoyl peroxide) can be added prior to oxidation.

Some oxidative methods of reducing recalcitrance in a biomass feedstock employ Fenton-type chemistry. Such methods are disclosed, for example, in U.S. Ser. No. 12/639,289, the complete disclosure of which is incorporated herein by reference.

Exemplary oxidants include peroxides, such as hydrogen peroxide and benzoyl peroxide, persulfates, such as ammonium persulfate, activated forms of oxygen, such as ozone, permanganates, such as potassium permanganate, perchlorates, such as sodium perchlorate, and hypochlorites, such as sodium hypochlorite (household bleach).

In some situations, pH is maintained at or below about 5.5 during contact, such as between 1 and 5, between 2 and 5, between 2.5 and 5 or between about 3 and 5. Oxidation conditions can also include a contact period of between 2 and 12 hours, e.g., between 4 and 10 hours or between 5 and 8 hours. In some instances, temperature is maintained at or below 300° C., e.g., at or below 250, 200, 150, 100 or 50° C. In some instances, the temperature remains substantially ambient, e.g., at or about 20-25° C.

In some embodiments, the one or more oxidants are applied as a gas, such as by generating ozone in-situ by irradiating the material through air with a beam of particles, such as electrons.

In some embodiments, the mixture further includes one or more hydroquinones, such as 2,5-dimethoxyhydroquinone (DMHQ) and/or one or more benzoquinones, such as 2,5-dimethoxy-1,4-benzoquinone (DMBQ), which can aid in electron transfer reactions.

In some embodiments, the one or more oxidants are electrochemically-generated in-situ. For example, hydrogen peroxide and/or ozone can be electro-chemically produced within a contact or reaction vessel.

Other Processes to Solubilize. Reduce Recalcitrance or to Functionalize

Any of the processes of this paragraph can be used alone without any of the processes described herein, or in combination with any of the processes described herein (in any order): steam explosion, chemical treatment (e.g., acid treatment (including concentrated and dilute acid treatment with mineral acids, such as sulfuric acid, hydrochloric acid and organic acids, such as trifluoroacetic acid) and/or base treatment (e.g., treatment with lime or sodium hydroxide)), UV treatment, screw extrusion treatment (see, e.g., U.S. Ser. No. 13/099,151, solvent treatment (e.g., treatment with ionic liquids) and freeze milling (see, e.g., U.S. Ser. No. 12/502,629 now U.S. Pat. No. 7,900,857).

Production of Fuels, Acids, Esters and/or Other Products and Uses

A typical feedstock obtained at least in part from plants contains cellulose, hemicellulose, and lignin plus lesser amounts of proteins, extractables and minerals. After one or more of the processing steps discussed above have been performed on the feedstock, the complex carbohydrates contained in the cellulose and hemicellulose fractions can in some cases be processed into fermentable sugars, optionally, along with acid or enzymatic hydrolysis. The sugars liberated can be converted into a variety of products, such as alcohols or organic acids. The product obtained depends upon the microorganism utilized and the conditions under which the bioprocessing occurs. In other embodiments, the treated feedstock can be subjected to thermochemical conversion, or other processing.

Examples of methods of further processing the treated feedstock are discussed in the following sections.

### Saccharification

In order to convert the treated feedstock to a form that can be readily fermented, in some implementations the cellulose in the feedstock is first hydrolyzed to low molecular weight carbohydrates, such as sugars, by a saccharifying agent, e.g., an enzyme, a process referred to as saccharification. In some implementations, the saccharifying agent comprises an acid, e.g., a mineral acid. When an acid is used, co-products may be generated that are toxic to microorganisms, in which case the process can further include removing such co-products. Removal may be performed using an activated carbon, e.g., activated charcoal, or other suitable techniques.

The treated feedstock can be hydrolyzed using an enzyme, e.g., by combining the material and the enzyme in a solvent, e.g., in an aqueous solution.

Enzymes and biomass-destroying organisms that break down biomass, such as the cellulose and/or the lignin portions of the feedstock, contain or manufacture various cellulolytic enzymes (cellulases), ligninases or various small molecule biomass-destroying metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (β-glucosidases). A cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally cellobiase cleaves cellobiose to yield glucose.

### Fermentation

Microorganisms can produce a number of useful intermediates and products by fermenting a low molecular weight sugar produced by saccharifying the treated feedstock. For example, fermentation or other bioprocesses can produce alcohols, organic acids, hydrocarbons, hydrogen, proteins or mixtures of any of these materials.

Yeast and Zymomonas bacteria, for example, can be used for fermentation or conversion. Other microorganisms are discussed in the Materials section, below. The optimum pH for fermentations is about pH 4 to 7. The optimum pH for yeast is from about pH 4 to 5, while the optimum pH for Zymomonas is from about pH 5 to 6. Typical fermentation times are about 24 to 168 (e.g., 24-96 hrs) hours with temperatures in the range of 20° C. to 40° C. (e.g., 26° C. to 40° C.), however thermophilic microorganisms prefer higher temperatures.

In some embodiments e.g., when anaerobic organisms are used, at least a portion of the fermentation is conducted in the absence of oxygen e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic condition can be achieved or maintained by carbon dioxide production during the fermentation and no additional inert gas is needed.

In some embodiments, all or a portion of the fermentation process can be interrupted before the low molecular weight sugar is completely converted to a product (e.g. ethanol). The intermediate fermentation products include high concentrations of sugar and carbohydrates. The sugars and carbohydrates can be isolated as discussed below. These intermediate fermentation products can be used in preparation of food for human or animal consumption. Additionally or alternatively, the intermediate fermentation products can be ground to a fine particle size in a stainless-steel laboratory mill to produce a flour-like substance.

The fermentations include the methods and products that are disclosed in U.S. application Ser. No. 14/016,471 filed Sep. 3, 2013, U.S. patent application Ser. No. 14/016,484 filed September 3, and U.S. application Ser. No. 14/016,481 filed Sep. 3, 2013, the entire disclosure described in these three applications is incorporated herein by reference.

Mobile fermenters can be utilized, as described in U.S. Provisional Patent Application Ser. No. 60/832,735, now Published International Application No. WO 2008/011598. Similarly, the saccharification equipment can be mobile. Further, saccharification and/or fermentation may be performed in part or entirely during transit.

### Fuel Cells

Where the methods described herein produce a sugar solution or suspension, this solution or suspension can subsequently be used in a fuel cell. For example, fuel cells utilizing sugars derived from cellulosic or lignocellulosic materials are disclosed in U.S. application Ser. No. 14/016,477 filed on Sep. 3, 2013, the complete disclosure of which is incorporated herein by reference.

### Thermochemical Conversion

Thermochemical conversion can be performed on the treated feedstock to produce one or more desired intermediates and/or products. A thermochemical conversion process includes changing molecular structures of carbon-containing material at elevated temperatures. Specific examples include gasification, pyrolysis, reformation, partial oxidation and mixtures of these (in any order).

Gasification converts carbon-containing materials into a synthesis gas (syngas), which can include methanol, carbon monoxide, carbon dioxide and hydrogen. Many microorganisms, such as acetogens or homoacetogens are capable of utilizing a syngas from the thermochemical conversion of biomass, to produce a product that includes an alcohol, a carboxylic acid, a salt of a carboxylic acid, a carboxylic acid ester or a mixture of any of these. Gasification of biomass (e.g., cellulosic or lignocellulosic materials), can be accomplished by a variety of techniques. For example, gasification can be accomplished utilizing staged steam reformation with a fluidized-bed reactor in which the carbonaceous material is first pyrolyzed in the absence of oxygen and then the pyrolysis vapors are reformed to synthesis gas with steam providing added hydrogen and oxygen. In such a technique, process heat comes from burning char. Another technique utilizes a screw auger reactor in which moisture and oxygen are introduced at the pyrolysis stage and the process heat is generated from burning some of the gas produced in the latter stage. Another technique utilizes entrained flow reformation in which both external steam and air are introduced in a single-stage gasification reactor. In partial oxidation gasification, pure oxygen is utilized with no steam.

### Post-Processing

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Other Possible Processing of Sugars

Processing during or after saccharification can include isolation and/or concentration of sugars by chromatography e.g., simulated moving bed chromatography, precipitation, centrifugation, crystallization, solvent evaporation and combinations thereof. In addition, or optionally, processing can include isomerization of one or more of the sugars in the sugar solution or suspension. Additionally, or optionally, the sugar solution or suspension can be chemically processed e.g., glucose and xylose can be hydrogenated to sorbitol and xylitol respectively. Hydrogenation can be accomplished by use of a catalyst e.g., Pt/γ-Al₂O₃, Ru/C, Raney Nickel in combination with H₂ under high pressure e.g., 10 to 12000 psi.

Some possible processing steps are disclosed in U.S. application Ser. No. 14/016,471 filed on Sep. 3, 2013, U.S. application Ser. No. 14/016,484 filed on Sep. 3, 2013, and in U.S. application Ser. No. 14/016,481 filed on Sep. 3, 2013, all three of which are incorporated by reference herein.

### Intermediates and Products

Using, e.g., such primary processes and/or post-processing, the treated biomass can be converted to one or more products, such as energy, fuels, foods and materials. Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols, e.g., containing greater than 10%, 20%, 30% or even greater than 40% water, sugars, biodiesel, organic acids (e.g., acetic acid and/or lactic acid), hydrocarbons, co-products (e.g., proteins, such as cellulolytic proteins (enzymes) or single cell proteins), and mixtures of any of these in any combination or relative concentration, and optionally in combination with any additives, e.g., fuel additives. Other examples include carboxylic acids, such as acetic acid or butyric acid, salts of a carboxylic acid, a mixture of carboxylic acids and salts of carboxylic acids and esters of carboxylic acids (e.g., methyl, ethyl and n-propyl esters), ketones, aldehydes, alpha, beta unsaturated acids, such as acrylic acid, olefins, such as ethylene, and mixtures of any of these. Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), methyl or ethyl esters of any of these alcohols. Other products include methyl acrylate, methylmethacrylate, lactic acid, propionic acid, butyric acid, succinic acid, 3-hydroxypropionic acid, a salt of any of the acids and a mixture of any of the acids and respective salts.

In some embodiments using, e.g., such primary processes and/or post-processing, the treated biomass can be converted to a platform chemical. For example, as stated above, the treated biomass can be converted to butanols (e.g., isobutanol, sec-butanol, tert-butanol or n-butanol) which are important platform chemicals. For example, dehydration of butanols can produce butenes such as 1-butene, cis-2-butene, trans-2-butene and isobutene, which are highly valuable starting materials for synthetic fuels, lubricants and other valuable chemicals. Specifically, 1-butene can be used in the creations of polymers, e.g., linear low density polyethylene, 2-butene isomers are valuable starting materials for lubricants and agricultural chemicals, and Isobutene can be polymerized to butyl rubber, methyl tert-butyl ether and isooctane. In addition, synthetic petroleum kerosene can be synthesized by oligomerization of butenes. Other intermediates and products, including food and pharmaceutical products, for example edible materials selected from the group consisting of pharmaceuticals, nutriceuticals, proteins, fats, vitamins, oils, fiber, minerals, sugars, carbohydrates and alcohols, are described in U.S. Ser. No. 12/417,900, the full disclosure of which is hereby incorporated by reference herein.

### Materials

### Modified Plant Materials

The plant feedstock is obtained at least in part from one or more types of modified plants, as discussed herein. In some cases, the feedstock includes more than one type of plant, and/or more than one portion of the plant, e.g., the stalk, fruit, and cob of a corn plant. The plant may be, for example, a corn, soybean, beet, cotton, rapeseed, potato, rice, alfalfa, or sugarcane plant. The plant may also be any of the many types of genetically modified plants that are grown. The feedstock may contain a mixture of different types of plants, different parts of a particular plant, and/or mixtures of plant materials with other materials e.g., biomass materials.

In some cases the entire plant can be used. For example, in cases where a crop is ruined by adverse growing conditions (e.g., drought, frost, flooding, pest infestation) the ruined crop can be useful in the methods and processes described herein.

### Other Feedstock Materials

In addition or as an alternative to the modified plant materials discussed above, the feedstock can include other materials e.g., biomass materials, that may or may not be genetically modified. The biomass can be, e.g., a cellulosic or lignocellulosic material. Such materials include paper and paper products (e.g., polycoated paper and Kraft paper), wood, wood-related materials, e.g., particle board, grasses, rice hulls, bagasse, jute, hemp, flax, bamboo, sisal, abaca, straw, switchgrass, alfalfa, hay, corn cobs, corn stover, coconut hair; and materials high in α-cellulose content, e.g., cotton. Feedstocks can be obtained from virgin scrap textile materials, e.g., remnants, post consumer waste, e.g., rags. When paper products are used they can be virgin materials, e.g., scrap virgin materials, or they can be post-consumer waste. Aside from virgin raw materials, post-consumer, industrial (e.g., offal), and processing waste (e.g., effluent from paper processing) can also be used as fiber sources. Biomass feedstocks can also be obtained or derived from human (e.g., sewage), animal or plant wastes. Additional cellulosic and lignocellulosic materials have been described in U.S. Pat. Nos. 6,448,307; 6,258,876; 6,207,729; 5,973.035 and 5,952,105.

In some embodiments, the biomass material includes a carbohydrate that is or includes a material having one or more β-1,4-linkages and having a number average molecular weight between about 3,000 and 50,000. Such a carbohydrate is or includes cellulose (I), which is derived from (β-glucose 1) through condensation of β(1,4)-glycosidic bonds. This linkage contrasts itself with that for α(1,4)-glycosidic bonds present in starch and other carbohydrates.

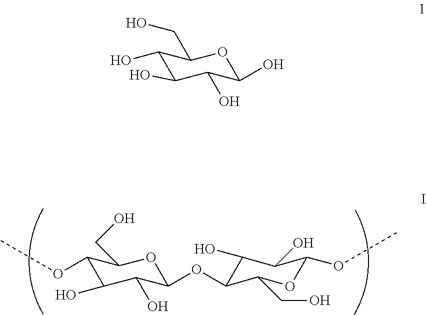

Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials.

In some instances the biomass is a microbial material. Microbial sources include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture systems.

### Saccharifying Agents

Suitable enzymes include cellobiases and cellulases capable of degrading biomass.

Suitable cellobiases include a cellobiase from Aspergillus niger sold under the tradename NOVOZYME™ 188.

Cellulases are capable of degrading biomass, and may be of fungal or bacterial origin. Suitable enzymes include cellulases from the genera Bacillus, Pseudomonas, Humicola, Fusarium, Thielavia, Acremonium, Chrysosporium and Trichoderma, and include species of Humicola, Coprinus, Thielavia, Fusarium, Myceliophthora, Acremonium, Cephalosporium, Scytalidium, Penicillium or Aspergillus (see, e.g., EP 458162), especially those produced by a strain selected from the species Humicola insolens (reclassified as Scytalidium thermophilum, see, e.g., U.S. Pat. No. 4,435,307), Coprinus cinereus, Fusarium oxysporum, Myceliophthora thermophile, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophile CBS 117.65, Cephalosporium sp. RYM-202, Acremonium sp. CBS 478.94, Acremonium sp. CBS 265.95, Acremonium persicinum CBS 169.65, Acremonium acremonium AHU 9519. Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H₂. Cellulolytic enzymes may also be obtained from Chrysosporium, preferably a strain of Chrysosporium lucknowense. Additionally, Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, e.g., EP 458162) may be used.

Enzyme complexes may be utilized, such as those available from GENENCORE® under the tradename ACCELLERASE®, for example, ACCELLERASE® 1500 enzyme complex. ACCELLERASE® 1500 enzyme complex contains multiple enzyme activities, mainly exoglucanase, endoglucanase (2200-2800 CMC U/g), hemi-cellulase, and beta-glucosidase (525-775 pNPG U/g), and has a pH of 4.6 to 5.0. The endoglucanase activity of the enzyme complex is expressed in carboxymethylcellulose activity units (CMC U), while the beta-glucosidase activity is reported in pNP-glucoside activity units (pNPG U). In one embodiment, a blend of ACCELLERASE® 1500 enzyme complex and NOVOZYME™ 188 cellobiase is used.

### Fermentation Agents

The microorganism(s) used in fermentation can be natural microorganisms and/or engineered microorganisms. For example, the microorganism can be a bacterium, e.g., a cellulolytic bacterium, a fungus, e.g., a yeast, a plant or a protist, e.g., an algae, a protozoa or a fungus-like protist, e.g., a slime mold. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Saccharomyces spp. e.g., Saccharomyces cerevisiae (baker's yeast), Saccharomyces distaticus, Saccharomyces uvarum; the genus Kluyveromyces, e.g., species Kluyveromyces marxianus, Kluyveromyces fragilis; the genus Candida, e.g., Candida pseudotropicalis, and Candida brassicae, Pichia stipitis (a relative of Candida shehatae, the genus Clavispora, e.g., species Clavispora lusitaniae and Clavispora opuntiae, the genus Pachysolen, e.g., species Pachysolen tannophilus, the genus Bretannomyces, e.g., species Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212). Other suitable microorganisms include, for example, Zymomonas mobilis, Clostridium thermocellum (Philippidis, 1996, supra), Clostridium saccharobutylacetonicum, Clostridium saccharobutylicum, Clostridium Puniceum, Clostridium beijernckii, Clostridium acetobutylicum, Moniliella pollinis, Yarrowia lipolytica, Aureobasidium sp., Trichosporonoides sp., Trigonopsis variabilis, Trichosporon sp., Moniliellaacetombutans, Typhula variabilis, Candida magnoliae, Ustilaginomycetes, Pseudozyma tsukubaensis, yeast species of genera Zygosaccharomyces, Debaromyces, Hansenula and Pichia, and fungi of the dematioid genus Torula.

Commercially available yeasts include, for example, RED STAR®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Other Embodiments

A number of embodiments of the invention have been described. Nevertheless, it will be understood that various modifications may be made without departing from the spirit and scope of the invention.

For example, the process parameters of any of the processing steps discussed herein can be adjusted based on the lignin content of the feedstock, for example as disclosed in U.S. Ser. No. 12/704,519, the full disclosure of which is incorporated herein by reference.

The process may include any of the features described in U.S. application Ser. No. 13/276,192, the full disclosure of which is incorporated herein by reference, including treating a cellulosic or lignocellulosic material to alter the structure of the material by irradiating the material with relatively low voltage, high power electron beam radiation, boiling or steeping the feedstock prior to saccharification, and irradiating a cellulosic or lignocellulosic material with an electron beam at a dose rate of at least 0.5 Mrad/sec.

While it is possible to perform all the processes described herein at one physical location, in some embodiments, the processes are completed at multiple sites, and/or may be performed during transport.

Lignin liberated in any process described herein can be captured and utilized. For example, the lignin can be used as captured as a plastic, or it can be synthetically upgraded to other plastics. In some instances, it can be utilized as an energy source, e.g., burned to provide heat. In some instances, it can also be converted to lignosulfonates, which can be utilized as binders, dispersants, emulsifiers or as sequestrants. Measurement of the lignin content of the starting feedstock can be used in process control in such lignin-capturing processes.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

As a dispersant, the lignin or lignosulfonates can be used, e.g., concrete mixes, clay and ceramics, dyes and pigments, leather tanning and in gypsum board.

As an emulsifier, the lignin or lignosulfonates can be used. e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

As a heating source, lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

Accordingly, other embodiments are within the scope of the following claims.

### Examples of Genetically Modified Plants

The following US Patents and US Patent applications disclose, by example, genetically modified material (e.g., plants, parts of plants) for the processes described herein or together with any materials described herein.

| | | | | | |
|---|---|---|---|---|---|
| 7,566,817 | 7,763,783 | 7,714,209 | 7,659,459 | 7,615,694 | 7,534,943 |
| 7,652,202 | 7,763,782 | 7,714,208 | 7,659,458 | 7,615,693 | 7,531,724 |
| 7,569,747 | 7,763,780 | 7,709,712 | 7,659,457 | 7,615,692 | 7,528,305 |
| 7,405,344 | 7,759,563 | 7,709,711 | 7,659,456 | 7,612,268 | 7,528,304 |
| 7,683,237 | 7,759,562 | 7,709,710 | 7,659,455 | 7,612,267 | 7,525,029 |
| 7,615,621 | 7,759,561 | 7,709,709 | 7,655,849 | 7,612,266 | 7,525,027 |
| 7,816,591 | 7,759,560 | 7,709,708 | 7,655,847 | 7,612,260 | 7,525,026 |
| 7,816,590 | 7,759,559 | 7,705,221 | 7,655,846 | 7,608,765 | 7,521,614 |
| 7,816,589 | 7,750,215 | 7,705,220 | 7,655,845 | 7,608,763 | 7,521,613 |
| 7,816,587 | 7,745,707 | 7,705,216 | 7,655,844 | 7,608,762 | 7,521,612 |
| 7,807,904 | 7,741,547 | 7,700,859 | 7,655,841 | 7,605,316 | 7,521,609 |
| 7,807,903 | 7,741,546 | 7,700,858 | 7,642,433 | 7,605,315 | 7,521,607 |
| 7,807,902 | 7,737,348 | 7,700,857 | 7,642,432 | 7,605,314 | 7,518,044 |
| 7,807,901 | 7,737,347 | 7,692,077 | 7,642,431 | 7,605,313 | 7,518,043 |
| 7,807,900 | 7,737,346 | 7,692,076 | 7,642,430 | 7,605,312 | 7,518,042 |
| 7,807,899 | 7,737,345 | 7,687,689 | 7,642,429 | 7,605,311 | 7,518,041 |
| 7,807,898 | 7,737,344 | 7,683,243 | 7,642,428 | 7,605,309 | 7,514,612 |
| 7,807,897 | 7,737,343 | 7,683,242 | 7,638,694 | 7,601,900 | 7,514,611 |
| 7,807,896 | 7,732,685 | 7,683,241 | 7,638,693 | 7,601,899 | 7,514,610 |
| 7,807,895 | 7,732,684 | 7,683,239 | 7,638,692 | 7,598,441 | 7,514,609 |
| 7,807,894 | 7,728,208 | 7,678,976 | 7,638,691 | 7,598,440 | 7,511,204 |
| 7,807,893 | 7,723,589 | 7,678,975 | 7,638,690 | 7,595,440 | 7,511,203 |
| 7,807,892 | 7,723,588 | 7,678,974 | 7,638,689 | 7,595,439 | 7,511,202 |
| 7,807,891 | 7,723,587 | 7,678,973 | 7,632,995 | 7,595,438 | 7,511,201 |
| 7,807,890 | 7,723,586 | 7,678,972 | 7,632,994 | 7,595,437 | 7,511,200 |
| 7,807,889 | 7,723,585 | 7,678,971 | 7,632,990 | 7,592,527 | 7,507,880 |
| 7,807,888 | 7,718,870 | 7,678,970 | 7,629,519 | 7,592,526 | 7,507,879 |
| 7,807,887 | 7,718,869 | 7,678,969 | 7,629,518 | 7,592,525 | 7,504,569 |
| 7,804,011 | 7,718,868 | 7,678,968 | 7,629,517 | 7,592,521 | 7,504,567 |
| 7,804,010 | 7,718,867 | 7,678,967 | 7,629,516 | 7,592,520 | 7,504,566 |
| 7,804,009 | 7,718,866 | 7,678,966 | 7,629,515 | 7,582,434 | 7,501,565 |
| 7,804,008 | 7,718,865 | 7,674,961 | 7,626,101 | 7,576,265 | 7,501,564 |
| 7,804,007 | 7,718,864 | 7,671,256 | 7,626,100 | 7,566,822 | 7,498,494 |
| 7,804,006 | 7,718,863 | 7,667,113 | 7,626,099 | 7,563,966 | 7,495,157 |
| 7,804,005 | 7,718,862 | 7,667,112 | 7,626,098 | 7,563,965 | 7,495,156 |
| 7,804,004 | 7,718,861 | 7,667,111 | 7,622,660 | 7,560,625 | 7,495,155 |
| 7,804,003 | 7,718,860 | 7,667,110 | 7,622,659 | 7,557,279 | 7,488,874 |
| 7,804,002 | 7,718,859 | 7,667,109 | 7,619,153 | 7,550,655 | 7,488,873 |
| 7,804,001 | 7,714,216 | 7,663,037 | 7,619,152 | 7,547,827 | 7,488,872 |
| 7,804,000 | 7,714,215 | 7,663,036 | 7,619,151 | 7,547,826 | 7,485,783 |
| 7,803,999 | 7,714,214 | 7,663,035 | 7,619,150 | 7,547,824 | 7,479,589 |
| 7,786,359 | 7,714,213 | 7,663,034 | 7,619,147 | 7,544,868 | 7,479,586 |
| 7,781,651 | 7,714,212 | 7,659,462 | 7,615,697 | 7,544,867 | 7,479,585 |
| 7,781,650 | 7,714,211 | 7,659,461 | 7,615,696 | 7,541,527 | 7,476,785 |
| 7,772,469 | 7,714,210 | 7,659,460 | 7,615,695 | 7,541,523 | 7,476,784 |
| 7,476,783 | 7,381,870 | 7,262,348 | 7,179,972 | 7,737,332 | 7,544,863 |
| 7,473,830 | 7,381,869 | 7,259,303 | 7,179,969 | 7,732,679 | 7,544,862 |
| 7,473,829 | 7,381,868 | 7,256,335 | 7,176,365 | 7,728,206 | 7,534,939 |
| 7,473,826 | 7,375,266 | 7,256,334 | 7,173,172 | 7,718,854 | 7,531,718 |
| 7,470,839 | 7,375,265 | 7,256,333 | 7,169,983 | 7,714,202 | 7,528,246 |
| 7,470,836 | 7,371,948 | 7,250,564 | 7,166,778 | 7,714,201 | 7,521,594 |
| 7,468,477 | 7,371,947 | 7,247,777 | 7,166,777 | 7,709,706 | 7,511,130 |
| 7,462,765 | 7,371,946 | 7,241,944 | 7,166,776 | 7,709,705 | 7,465,849 |
| 7,462,764 | 7,371,945 | 7,235,726 | 7,164,068 | 7,388,135 | 7,423,203 |
| 7,462,763 | 7,368,643 | 7,235,725 | 7,164,065 | 7,388,134 | 7,417,177 |
| 7,459,613 | 7,368,640 | 7,235,724 | 7,161,070 | 7,381,861 | 7,417,176 |
| 7,459,612 | 7,365,252 | 7,235,722 | 7,161,069 | 7,253,345 | 7,408,096 |
| 7,459,611 | 7,365,251 | 7,235,720 | 7,157,630 | 7,250,563 | 7,405,343 |
| 7,456,345 | 7,365,250 | 7,232,945 | 7,157,626 | 7,247,774 | 7,385,107 |
| 7,456,344 | 7,365,249 | 7,232,944 | 7,157,625 | 7,223,907 | 7,365,241 |
| 7,456,343 | 7,361,820 | 7,232,943 | 7,157,624 | 7,189,514 | 7,335,812 |
| 7,456,342 | 7,361,819 | 7,230,173 | 7,157,281 | RE39,580 | 7,329,799 |
| 7,453,031 | 7,361,818 | 7,227,062 | 7,154,031 | 7,816,581 | 7,304,206 |
| 7,453,030 | 7,361,817 | 7,227,061 | 7,151,208 | 7,812,219 | 7,294,711 |
| 7,449,622 | 7,361,815 | 7,223,908 | 7,148,410 | 7,807,874 | 7,288,408 |
| 7,449,621 | 7,361,814 | 7,220,900 | 6,906,250 | 7,807,873 | 7,268,276 |
| 7,449,620 | 7,358,427 | 7,217,874 | 6,864,409 | 7,807,812 | 7,262,339 |
| 7,449,619 | 7,355,107 | 7,217,873 | 6,855,877 | 7,807,811 | 7,250,501 |
| 7,439,424 | 7,351,890 | 7,217,872 | 6,825,400 | 7,803,928 | 7,244,877 |
| 7,432,427 | 7,351,888 | 7,217,871 | 6,114,610 | 7,799,970 | 7,230,165 |
| 7,432,426 | 7,342,156 | 7,211,717 | 6,103,959 | 7,790,953 | 7,227,056 |
| 7,429,696 | 7,342,155 | 7,211,716 | 6,103,958 | 7,786,353 | 7,217,867 |
| 7,423,207 | 7,342,152 | 7,208,663 | 6,084,161 | 7,786,350 | 7,217,865 |
| 7,423,206 | 7,339,101 | 7,208,662 | 6,054,640 | 7,750,207 | 7,205,457 |
| 7,423,204 | 7,339,100 | 7,208,661 | 7,112,725 | 7,745,694 | 7,195,917 |
| 7,417,183 | 7,339,099 | 7,208,660 | 7,825,304 | 7,728,190 | 7,186,893 |
| 7,417,182 | 7,339,098 | 7,205,466 | 7,825,303 | 7,714,189 | 7,157,619 |
| 7,414,181 | 7,335,827 | 7,205,465 | 7,825,302 | 7,705,201 | 7,151,204 |
| 7,408,099 | 7,335,826 | 7,205,464 | 7,825,301 | 7,700,838 | 7,148,398 |
| 7,399,915 | 7,335,822 | 7,199,291 | 7,825,300 | 7,692,067 | 7,141,722 |
| 7,399,912 | 7,329,803 | 7,199,290 | 7,825,300 | 7,674,952 | 7,138,278 |
| 7,399,911 | 7,321,088 | 7,193,146 | 7,820,888 | 7,674,894 | 7,122,719 |
| 7,394,003 | 7,321,087 | 7,193,143 | 7,820,887 | 7,662,940 | 7,112,717 |
| 7,390,946 | 7,321,086 | 7,189,906 | 7,803,997 | 7,655,838 | 7,078,592 |
| 7,390,945 | 7,321,085 | 7,189,904 | 7,799,972 | 7,635,764 | 7,067,722 |
| 7,388,140 | 7,321,084 | 7,189,903 | 7,750,213 | 7,625,738 | 7,064,249 |
| 7,388,139 | 7,319,182 | 7,186,906 | 7,750,212 | 7,615,680 | 7,022,897 |
| 7,385,122 | 7,317,155 | 7,186,904 | 7,745,704 | 7,605,244 | 6,943,281 |
| 7,385,121 | 7,297,848 | 7,186,903 | 7,741,543 | 7,601,890 | 6,916,970 |
| 7,385,120 | 7,294,772 | 7,186,901 | 7,737,335 | 7,595,382 | 6,841,717 |
| 7,381,874 | 7,288,704 | 7,186,899 | 7,737,334 | 7,589,188 | 6,822,142 |
| 7,381,873 | 7,268,279 | 7,183,471 | 7,737,333 | 7,553,952 | 6,803,501 |
| 6,620,988 | 7,728,196 | 7,626,089 | 7,557,277 | 7,491,870 | 7,381,867 |
| 6,538,179 | 7,723,583 | 7,626,088 | 7,567,276 | 7,488,869 | 7,381,866 |
| 6,538,178 | 7,714,198 | 7,626,087 | 7,557,275 | 7,488,868 | 7,378,578 |
| 6,501,009 | 7,709,703 | 7,626,086 | 7,557,274 | 7,488,867 | 7,378,577 |
| 6,476,295 | 7,705,211 | 7,622,646 | 7,557,273 | 7,485,781 | 7,375,262 |
| 6,448,476 | 7,705,208 | 7,622,645 | 7,557,272 | 7,485,780 | 7,371,938 |
| 6,448,473 | 7,705,207 | 7,622,644 | 7,557,271 | 7,462,516 | 7,368,637 |
| 6,284,949 | 7,700,849 | 7,622,643 | 7,557,270 | 7,479,583 | 7,368,635 |
| 6,281,016 | 7,700,847 | 7,622,642 | 7,554,016 | 7,479,582 | 7,358,420 |
| 6,177,615 | 7,700,846 | 7,619,143 | 7,554,015 | 7,468,474 | 7,355,103 |
| 6,175,061 | 7,700,844 | 7,619,142 | 7,554,014 | 7,459,609 | 7,355,102 |
| 6,156,573 | 7,700,843 | 7,619,141 | 7,554,013 | 7,453,029 | 7,351,886 |
| 6,107,549 | 7,692,070 | 7,619,140 | 7,550,653 | 7,453,028 | 7,351,885 |
| 6,023,013 | 7,687,686 | 7,619,139 | 7,531,722 | 7,446,244 | 7,345,228 |
| 5,463,175 | 7,687,685 | 7,615,688 | 7,531,721 | 7,442,864 | 7,345,227 |
| 7,531,725 | 7,687,684 | 7,615,687 | 7,531,720 | 7,442,863 | 7,345,226 |
| 7,468,476 | 7,678,965 | 7,612,259 | 7,531,719 | 7,442,862 | 7,345,225 |
| 7,253,346 | 7,678,964 | 7,608,761 | 7,528,306 | 7,442,860 | 7,345,224 |
| 7,214,863 | 7,678,963 | 7,605,306 | 7,528,301 | 7,439,422 | 7,342,151 |
| 7,186,900 | 7,678,962 | 7,598,434 | 7,525,028 | 7,423,200 | 7,342,150 |
| 7,166,780 | 7,659,454 | 7,595,435 | 7,525,025 | 7,423,199 | 7,332,656 |
| 7,166,779 | 7,659,453 | 7,592,517 | 7,525,019 | 7,414,177 | 7,332,655 |
| 7,157,628 | 7,659,452 | 7,592,516 | 7,525,018 | 7,414,176 | 7,329,801 |
| 7,157,627 | 7,655,839 | 7,592,514 | 7,525,017 | 7,408,097 | 7,326,832 |
| 7,563,949 | 7,652,199 | 7,592,513 | 7,521,608 | 7,405,349 | 7,321,082 |
| 7,807,884 | 7,652,198 | 7,592,512 | 7,521,605 | 7,405,348 | 7,321,079 |
| 7,799,973 | 7,652,197 | 7,592,511 | 7,518,036 | 7,399,909 | 7,314,983 |
| 7,790,964 | 7,649,129 | 7,562,810 | 7,514,607 | 7,399,907 | 7,314,982 |
| 7,786,357 | 7,649,128 | 7,579,525 | 7,514,606 | 7,396,983 | 7,314,981 |
| 7,781,649 | 7,649,127 | 7,579,524 | 7,514,605 | 7,394,000 | 7,314,980 |
| 7,777,104 | 7,649,126 | 7,579,523 | 7,514,604 | 7,390,942 | 7,312,382 |
| 7,777,103 | 7,642,413 | 7,572,960 | 7,514,603 | 7,390,941 | 7,312,380 |
| 7,767,887 | 7,642,412 | 7,572,958 | 7,514,602 | 7,390,940 | 7,309,818 |
| 7,759,556 | 7,642,411 | 7,572,957 | 7,507,878 | 7,390,939 | 7,307,201 |
| 7,759,553 | 7,642,410 | 7,572,956 | 7,507,877 | 7,390,938 | 7,304,218 |
| 7,759,551 | 7,642,409 | 7,569,752 | 7,504,565 | 7,388,132 | 7,304,214 |
| 7,732,677 | 7,642,408 | 7,569,751 | 7,504,564 | 7,388,131 | 7,304,213 |
| 7,732,676 | 7,642,407 | 7,569,750 | 7,501,563 | 7,388,130 | 7,301,076 |
| 7,732,675 | 7,632,987 | 7,566,821 | 7,501,562 | 7,385,117 | 7,297,843 |
| 7,732,674 | 7,632,985 | 7,566,820 | 7,501,560 | 7,385,116 | 7,294,770 |
| 7,732,673 | 7,629,510 | 7,563,955 | 7,498,491 | 7,385,115 | 7,294,768 |
| 7,732,672 | 7,629,509 | 7,563,954 | 7,498,490 | 7,385,113 | 7,294,765 |
| 7,728,204 | 7,629,508 | 7,563,953 | 7,498,489 | 7,385,112 | 7,294,764 |
| 7,728,203 | 7,629,507 | 7,560,619 | 7,498,486 | 7,385,111 | 7,294,763 |
| 7,728,202 | 7,629,506 | 7,560,618 | 7,498,485 | 7,385,110 | 7,291,771 |
| 7,728,201 | 7,626,091 | 7,560,617 | 7,498,484 | 7,385,109 | 7,291,769 |
| 7,728,199 | 7,626,090 | 7,560,616 | 7,491,871 | 7,385,108 | 7,285,704 |
| 7,279,621 | 7,132,591 | 7,045,687 | 6,900,373 | 6,080,916 | 5,902,923 |
| 7,276,648 | 7,129,399 | 7,045,686 | 6,900,372 | 6,063,990 | 5,898,100 |
| 7,271,324 | 7,126,046 | 7,041,881 | 6,894,207 | 6,063,989 | 5,880,346 |
| 7,265,277 | 7,119,260 | 7,041,880 | 6,888,049 | 6,051,761 | 5,880,345 |
| 7,265,276 | 7,119,259 | 7,041,879 | 6,888,048 | 6,043,414 | 5,880,344 |
| 7,265,275 | 7,119,258 | 7,038,114 | 6,884,927 | 6,040,499 | 5,872,304 |
| 7,265,274 | 7,115,801 | 7,034,210 | 6,884,926 | 6,037,529 | 5,872,303 |
| 7,265,273 | 7,109,399 | 7,034,209 | 6,884,925 | 6,034,303 | 5,866,774 |
| 7,265,272 | 7,105,728 | 7,030,301 | 6,884,924 | 6,034,302 | 5,866,773 |
| 7,265,271 | 7,106,727 | 7,030,300 | 6,884,923 | 6,034,301 | 5,866,772 |
| 7,265,270 | 7,106,726 | 7,019,199 | 6,881,879 | 6,034,300 | 5,866,771 |
| 7,259,299 | 7,102,062 | 7,012,174 | 6,875,908 | 6,034,299 | 5,859,352 |
| 7,259,298 | 7,102,061 | 7,005,563 | 6,870,079 | 6,031,159 | 5,723,745 |
| 7,256,330 | 7,098,385 | 7,002,062 | 6,861,579 | 6,020,542 | 7,268,274 |
| 7,247,772 | 7,098,384 | 7,002,058 | 6,858,784 | 6,020,541 | 7,402,731 |
| 7,247,771 | 7,091,403 | 6,982,367 | 6,858,783 | 6,018,108 | 6,865,556 |
| 7,244,881 | 7,087,815 | 6,982,366 | 6,852,912 | 6,018,107 | 5,424,412 |
| 7,241,941 | 7,084,328 | 6,979,761 | 6,849,786 | 6,005,171 | 5,463,175 |
| 7,241,939 | 7,084,326 | 6,979,760 | 6,849,785 | 6,005,170 | 5,484,956 |
| 7,235,718 | 7,084,325 | 6,972,355 | 6,846,973 | 6,002,073 | 5,554,798 |
| 7,217,870 | 7,084,324 | 6,972,354 | 6,835,873 | 5,998,709 | 559,387 |
| 7,217,869 | 7,081,572 | 6,972,353 | 6,828,489 | 5,998,708 | 5,641,876 |
| 7,217,868 | 7,078,600 | 6,969,787 | 6,815,589 | 5,988,707 | 5,659,122 |
| 7,196,253 | 7,078,598 | 6,967,263 | 6,815,588 | 5,998,706 | 571,084 |
| 7,196,252 | 7,078,597 | 6,960,707 | 6,815,587 | 5,998,705 | 5,728,925 |
| 7,196,251 | 7,078,595 | 6,958,436 | 6,815,586 | 5,998,704 | 5,750,871 |
| 7,193,140 | 7,074,989 | 6,953,876 | 6,809,237 | 5,998,703 | 5,804,425 |
| 7,193,139 | 7,074,983 | 6,951,973 | 6,781,040 | 5,990,391 | 5,859,347 |
| 7,193,137 | 7,071,390 | 6,936,754 | 6,653,534 | 5,986,179 | 6,020,190 |
| 7,189,900 | 7,071,389 | 6,936,753 | 6,198,027 | 5,986,178 | 6,025,545 |
| 7,189,898 | 7,071,388 | 6,933,423 | 6,177,618 | 5,981,851 | 6,040,497 |
| 7,183,467 | 7,071,387 | 6,924,418 | 6,169,227 | 5,981,850 | 6,051,753 |
| 7,183,465 | 7,067,723 | 6,919,498 | 6,137,034 | 5,981,849 | 6,180,774 |
| 7,183,464 | 7,064,253 | 6,914,174 | 6,133,510 | 5,981,848 | 6,218,188 |
| 7,183,463 | 7,060,878 | 6,914,173 | 6,124,527 | 5,981,845 | 6,340,593 |
| 7,183,462 | 7,060,877 | 6,914,172 | 6,121,518 | 5,977,449 | 6,489,542 |
| 7,183,461 | 7,057,096 | 6,911,585 | 6,121,517 | 5,977,448 | 6,501,009 |
| 7,176,359 | 7,057,095 | 6,911,581 | 6,121,516 | 5,977,447 | 6,548,291 |
| 7,176,358 | 7,057,094 | 6,911,580 | 6,121,515 | 5,977,444 | 6,573,240 |
| 7,176,357 | 7,057,093 | 6,911,579 | 6,114,604 | 5,973,235 | 6,645,497 |
| 7,173,168 | 7,057,092 | 6,911,578 | 6,103,957 | 5,969,218 | 6,660,911 |
| 7,169,976 | 7,053,280 | 6,906,248 | 6,100,454 | 5,969,217 | 6,737,273 |
| 7,169,975 | 7,063,279 | 6,906,247 | 6,096,949 | 5,945,588 | 6,753,463 |
| 7,169,974 | 7,053,272 | 6,906,246 | 6,091,005 | 5,942,666 | 6,825,400 |
| 7,166,774 | 7,049,494 | 6,903,253 | 6,087,562 | 5,932,786 | 6,893,872 |
| 7,148,408 | 7,049,493 | 6,903,251 | 6,084,159 | 5,929,310 | 6,900,371 |
| 7,138,570 | 7,045,691 | 6,900,376 | 6,080,918 | 5,907,088 | 6,943,282 |
| 6,949,696 | 7,482,510 | 7,834,247 | 7,772,465 | 7,663,031 | 7,601,894 |
| 6,962,705 | 7,473,819 | 7,834,246 | 7,772,370 | 7,663,029 | 7,598,443 |
| 7,064,249 | 7,465,850 | 7,834,245 | 7,767,889 | 7,655,848 | 7,598,442 |
| 7,112,665 | 7,456,337 | 7,834,240 | 7,767,888 | 7,655,843 | 7,598,439 |
| 7,112,725 | 7,456,335 | 7,829,764 | 7,763,778 | 7,655,842 | 7,598,438 |
| 7,141,722 | 7,442,853 | 7,829,760 | 7,763,465 | 7,652,201 | 7,598,437 |
| 7,157,281 | 7,439,417 | 7,825,310 | 7,759,564 | 7,652,200 | 7,598,435 |
| 7,223,907 | 7,435,875 | 7,825,309 | 7,759,555 | 7,652,195 | 7,595,436 |
| 7,227,056 | 7,427,698 | 7,825,308 | 7,759,554 | 7,645,923 | 7,592,524 |
| 7,250,501 | 7,427,696 | 7,825,307 | 7,759,544 | 7,645,922 | 7,592,523 |
| 7,288,643 | 7,425,666 | 7,825,299 | 7,759,543 | 7,645,921 | 7,592,522 |
| 7,381,861 | 7,425,665 | 7,825,294 | 7,754,949 | 7,642,421 | 7,592,519 |
| 7,435,807 | 7,423,196 | 7,825,234 | 7,754,948 | 7,642,420 | 7,592,505 |
| 7,449,564 | 7,399,904 | 7,820,895 | 7,750,216 | 7,642,419 | 7,589,264 |
| 7,514,544 | 7,399,903 | 7,820,894 | 7,745,706 | 7,642,418 | 7,589,263 |
| RE38825 | 7,375,209 | 7,820,893 | 7,745,705 | 7,642,417 | 7,589,261 |
| RE39247 | 7,317,140 | 7,820,892 | 7,745,702 | 7,638,695 | 7,589,260 |
| 7,829,761 | 7,303,919 | 7,820,891 | 7,745,701 | 7,638,688 | 7,589,259 |
| 7,807,882 | 7,271,316 | 7,820,886 | 7,741,545 | 7,632,993 | 7,589,258 |
| 7,803,987 | 7,259,294 | 7,820,885 | 7,737,342 | 7,632,992 | 7,589,257 |
| 7,799,971 | 7,238,856 | 7,816,586 | 7,737,341 | 7,632,989 | 7,589,176 |
| 7,795,500 | 7,235,713 | 7,816,585 | 7,737,340 | 7,632,988 | 7,586,028 |
| 7,795,414 | 7,220,585 | 7,812,231 | 7,737,336 | 7,629,514 | 7,586,027 |
| 7,790,873 | 7,189,693 | 7,812,230 | 7,737,330 | 7,629,513 | 7,586,026 |
| 7,763,777 | 7,186,561 | 7,812,226 | 7,736,897 | 7,629,512 | 7,586,025 |
| 7,763,776 | 7,179,962 | 7,812,225 | 7,732,683 | 7,629,511 | 7,582,816 |
| 7,718,858 | 7,176,026 | 7,812,223 | 7,732,668 | 7,629,505 | 7,582,815 |
| 7,718,857 | 7,166,767 | 7,812,216 | 7,728,207 | 7,629,504 | 7,582,814 |
| 7,714,190 | 7,164,057 | 7,807,883 | 7,718,856 | 7,624,533 | 7,582,813 |
| 7,709,698 | 7,161,063 | 7,807,876 | 7,714,205 | 7,622,647 | 7,582,812 |
| 7,652,203 | 7,135,618 | 7,803,998 | 7,714,187 | 7,622,637 | 7,582,811 |
| 7,622,570 | 7,125,719 | 7,803,996 | 7,714,184 | 7,619,149 | 7,582,808 |
| 7,619,137 | 7,105,723 | 7,803,993 | 7,709,702 | 7,619,148 | 7,579,530 |
| 7,608,759 | 7,087,261 | 7,803,990 | 7,705,219 | 7,615,690 | 7,579,529 |
| 7,608,757 | 7,034,208 | 7,803,989 | 7,705,218 | 7,612,265 | 7,579,522 |
| 7,598,431 | 6,867,351 | 7,799,977 | 7,700,856 | 7,612,264 | 7,576,271 |
| 7,579,517 | 6,825,399 | 7,799,975 | 7,700,855 | 7,612,263 | 7,576,270 |
| 7,563,948 | 6,818,805 | 7,799,974 | 7,700,854 | 7,612,262 | 7,576,269 |
| 7,521,598 | 6,784,338 | 7,799,566 | 7,700,836 | 7,612,256 | 7,576,268 |
| 7,521,597 | 6,774,288 | 7,795,508 | 7,700,832 | 7,612,254 | 7,576,267 |
| 7,514,599 | 6,720,477 | 7,795,506 | 7,692,061 | 7,612,251 | 7,576,266 |
| 7,504,559 | 6,710,229 | 7,790,969 | 7,687,687 | 7,608,764 | 7,572,963 |
| 7,498,482 | 6,689,939 | 7,790,874 | 7,683,240 | 7,608,755 | 7,572,962 |
| 7,498,429 | 6,677,504 | 7,777,107 | 7,667,115 | 7,608,752 | 7,572,961 |
| 7,495,151 | 6,329,518 | 7,777,106 | 7,667,107 | 7,605,307 | 7,572,955 |
| 7,485,775 | 6,225,526 | 7,772,468 | 7,663,033 | 7,601,898 | 7,569,757 |
| 7,482,511 | 7,834,257 | 7,772,467 | 7,663,032 | 7,601,897 | 7,569,756 |
| 7,569,755 | 7,525,023 | 7,456,339 | 7,371,936 | 7,317,149 | 7,276,650 |
| 7,569,754 | 7,525,022 | 7,442,861 | 7,365,253 | 7,317,148 | 7,276,649 |
| 7,569,753 | 7,525,021 | 7,439,425 | 7,361,812 | 7,317,147 | 7,276,647 |
| 7,569,749 | 7,525,020 | 7,439,421 | 7,361,807 | 7,317,146 | 7,276,596 |
| 7,566,819 | 7,521,611 | 7,439,348 | 7,358,425 | 7,317,145 | 7,273,975 |
| 7,563,964 | 7,521,610 | 7,435,885 | 7,358,424 | 7,317,143 | 7,273,973 |
| 7,563,963 | 7,521,604 | 7,435,883 | 7,358,423 | 7,317,137 | 7,273,972 |
| 7,563,962 | 7,521,603 | 7,435,881 | 7,355,108 | 7,314,990 | 7,273,971 |
| 7,563,961 | 7,521,602 | 7,435,880 | 7,355,106 | 7,314,989 | 7,273,965 |
| 7,563,960 | 7,521,601 | 7,435,879 | 7,355,105 | 7,314,988 | 7,271,327 |
| 7,563,959 | 7,518,037 | 7,432,424 | 7,355,104 | 7,314,987 | 7,271,326 |
| 7,563,958 | 7,514,601 | 7,432,423 | 7,351,882 | 7,312,385 | 7,271,323 |
| 7,563,957 | 7,511,205 | 7,432,422 | 7,351,878 | 7,312,384 | 7,271,319 |
| 7,560,624 | 7,511,196 | 7,432,421 | 7,348,469 | 7,312,377 | 7,270,380 |
| 7,560,623 | 7,511,195 | 7,432,418 | 7,348,468 | 7,312,375 | 7,268,278 |
| 7,560,612 | 7,511,194 | 7,429,695 | 7,345,230 | 7,309,816 | 7,268,277 |
| 7,557,266 | 7,511,193 | 7,427,702 | 7,342,157 | 7,306,946 | 7,268,270 |
| 7,557,263 | 7,511,192 | 7,427,701 | 7,342,154 | 7,304,222 | 7,268,226 |
| 7,554,020 | 7,511,188 | 7,427,700 | 7,339,097 | 7,304,221 | 7,265,279 |
| 7,553,951 | 7,504,568 | 7,423,202 | 7,339,096 | 7,304,212 | 7,265,265 |
| 7,550,657 | 7,504,558 | 7,423,197 | 7,339,092 | 7,304,211 | 7,262,350 |
| 7,550,656 | 7,501,561 | 7,420,103 | 7,335,828 | 7,301,082 | 7,262,349 |
| 7,550,575 | 7,498,488 | 7,414,180 | 7,335,825 | 7,301,080 | 7,262,347 |
| 7,547,832 | 7,498,487 | 7,414,179 | 7,335,824 | 7,301,079 | 7,262,346 |
| 7,547,831 | 7,498,413 | 7,414,174 | 7,335,823 | 7,301,075 | 7,262,345 |
| 7,547,830 | 7,495,154 | 7,411,118 | 7,335,817 | 7,301,069 | 7,262,342 |
| 7,547,829 | 7,495,150 | 7,411,113 | 7,332,660 | 7,297,850 | 7,259,305 |
| 7,547,825 | 7,491,869 | 7,411,112 | 7,332,659 | 7,297,849 | 7,259,304 |
| 7,547,822 | 7,485,779 | 7,399,914 | 7,332,658 | 7,297,841 | 7,259,302 |
| 7,544,869 | 7,485,778 | 7,399,910 | 7,332,650 | 7,294,774 | 7,259,301 |
| 7,544,866 | 7,482,515 | 7,399,908 | 7,329,806 | 7,294,769 | 7,256,332 |
| 7,544,865 | 7,482,513 | 7,399,906 | 7,329,805 | 7,294,767 | 7,256,331 |
| 7,544,864 | 7,479,588 | 7,396,980 | 7,329,804 | 7,294,766 | 7,256,322 |
| 7,544,857 | 7,479,581 | 7,393,999 | 7,326,836 | 7,291,774 | 7,256,280 |
| 7,541,526 | 7,476,781 | 7,388,141 | 7,326,835 | 7,291,773 | 7,253,000 |
| 7,541,525 | 7,473,828 | 7,388,137 | 7,326,833 | 7,288,703 | 7,250,552 |
| 7,541,524 | 7,473,827 | 7,388,133 | 7,326,830 | 7,288,701 | 7,241,943 |
| 7,541,521 | 7,473,821 | 7,388,128 | 7,323,623 | 7,288,700 | 7,241,942 |
| 7,541,520 | 7,470,838 | 7,388,125 | 7,321,089 | 7,288,699 | 7,241,940 |
| 7,541,517 | 7,470,834 | 7,381,872 | 7,321,083 | 7,285,707 | 7,241,934 |
| 7,538,261 | 7,470,833 | 7,381,871 | 7,321,031 | 7,285,706 | 7,238,859 |
| 7,528,308 | 7,468,278 | 7,381,865 | 7,319,183 | 7,285,702 | 7,235,723 |
| 7,528,307 | 7,465,856 | 7,381,863 | 7,317,154 | 7,282,629 | 7,232,946 |
| 7,528,300 | 7,465,852 | 7,378,574 | 7,317,153 | 7,282,627 | 7,230,172 |
| 7,528,299 | 7,462,766 | 7,375,264 | 7,317,152 | 7,282,626 | 7,230,171 |
| 7,528,293 | 7,462,760 | 7,375,263 | 7,317,151 | 7,279,615 | 7,230,169 |
| 7,525,024 | 7,459,610 | 7,371,944 | 7,317,150 | 7,276,652 | 7,230,158 |
| 7,227,065 | 7,179,971 | 7,151,207 | 7,064,255 | 7,005,565 | 6,951,974 |
| 7,227,064 | 7,179,970 | 7,151,205 | 7,064,252 | 7,002,061 | 6,949,699 |
| 7,227,063 | 7,179,968 | 7,148,406 | 7,064,251 | 7,002,056 | 6,946,589 |
| 7,227,060 | 7,179,967 | 7,148,401 | 7,064,250 | 6,998,518 | 6,943,279 |
| 7,227,059 | 7,179,963 | 7,141,721 | 7,064,247 | 6,995,305 | 6,936,756 |
| 7,227,058 | 7,179,955 | 7,129,402 | 7,060,879 | 6,995,304 | 6,936,755 |
| 7,220,902 | 7,179,599 | 7,129,401 | 7,060,813 | 6,995,303 | 6,936,752 |
| 7,220,901 | 7,176,364 | 7,129,395 | 7,053,286 | 6,992,240 | 6,936,751 |
| 7,214,865 | 7,176,363 | 7,122,725 | 7,053,285 | 6,992,239 | 6,933,427 |
| 7,214,864 | 7,176,362 | 7,115,802 | 7,053,284 | 6,992,238 | 6,933,425 |
| 7,214,860 | 7,176,360 | 7,112,731 | 7,053,283 | 6,992,237 | 6,930,230 |
| 7,214,857 | 7,176,356 | 7,112,729 | 7,053,282 | 6,989,481 | 6,930,229 |
| 7,214,855 | 7,176,349 | 7,112,728 | 7,053,275 | 6,989,480 | 6,930,225 |
| 7,214,854 | 7,176,027 | 7,109,403 | 7,049,499 | 6,989,479 | 6,927,327 |
| 7,214,852 | 7,173,174 | 7,109,391 | 7,049,495 | 6,989,478 | 6,927,326 |
| 7,211,718 | 7,173,173 | 7,109,390 | 7,045,692 | 6,989,475 | 6,924,421 |
| 7,211,714 | 7,169,988 | 7,102,064 | 7,045,682 | 6,989,474 | 6,921,852 |
| 7,211,712 | 7,169,987 | 7,102,063 | 7,041,887 | 6,987,217 | 6,921,850 |
| 7,205,455 | 7,169,986 | 7,098,390 | 7,041,886 | 6,987,212 | 6,921,847 |
| 7,205,453 | 7,169,985 | 7,098,381 | 7,041,874 | 6,964,778 | 6,919,500 |
| RE39,562 | 7,169,984 | 7,094,957 | 7,038,109 | 6,962,371 | 6,919,499 |
| 7,202,403 | 7,169,980 | 7,094,956 | 7,038,108 | 6,979,764 | 6,916,975 |
| 7,202,402 | 7,169,979 | 7,091,407 | 7,034,214 | 6,979,763 | 6,914,178 |
| 7,199,294 | 7,169,978 | 7,091,406 | 7,034,213 | 6,979,759 | 6,914,177 |
| 7,199,293 | 7,169,977 | 7,091,398 | 7,034,211 | 6,977,327 | 6,914,171 |
| 7,199,292 | 7,169,973 | 7,067,823 | 7,030,303 | 6,974,900 | 6,914,170 |
| 7,199,289 | 7,166,784 | 7,087,822 | 7,030,302 | 6,974,899 | 6,911,587 |
| 7,196,256 | 7,166,782 | 7,087,821 | 7,030,298 | 6,972,357 | 6,911,577 |
| 7,196,255 | 7,166,781 | 7,087,820 | 7,026,533 | 6,972,336 | 6,909,039 |
| 7,196,254 | 7,166,769 | 7,084,335 | 7,022,904 | 6,972,352 | 6,909,038 |
| 7,193,145 | 7,166,765 | 7,084,332 | 7,022,902 | 6,969,790 | 6,906,251 |
| 7,193,144 | 7,164,070 | 7,084,327 | 7,022,899 | 6,969,789 | 6,906,243 |
| 7,193,141 | 7,164,069 | 7,081,566 | 7,019,200 | 6,969,788 | 6,905,857 |
| 7,193,136 | 7,164,067 | 7,078,603 | 7,019,198 | 6,969,786 | 6,903,254 |
| 7,193,135 | 7,164,066 | 7,078,602 | 7,015,386 | 6,967,269 | 6,903,205 |
| 7,193,130 | 7,164,063 | 7,078,601 | 7,015,385 | 6,967,268 | 6,900,378 |
| 7,189,905 | 7,164,062 | 7,078,596 | 7,015,381 | 6,967,267 | 6,900,377 |
| 7,189,902 | 7,164,061 | 7,078,589 | 7,015,380 | 6,967,264 | 6,897,365 |
| 7,189,901 | 7,164,056 | 7,074,991 | 7,015,379 | 6,965,063 | 6,897,364 |
| 7,189,899 | 7,161,074 | 7,071,397 | 7,015,376 | 6,960,708 | 6,897,363 |
| 7,189,889 | 7,161,073 | 7,071,396 | 7,015,375 | 6,958,438 | 6,897,362 |
| 7,186,905 | 7,161,072 | 7,071,395 | 7,012,177 | 6,958,437 | 6,897,361 |
| 7,186,902 | 7,161,071 | 7,071,394 | 7,012,176 | 6,956,153 | 6,897,360 |
| 7,186,896 | 7,161,068 | 7,071,393 | 7,009,094 | 6,956,150 | 6,891,090 |
| 7,183,472 | 7,161,065 | 7,067,727 | 7,009,093 | 6,953,878 | 6,891,085 |
| 7,183,469 | 7,157,632 | 7,067,720 | 7,009,087 | 6,953,877 | 6,888,051 |
| 7,183,460 | 7,154,030 | 7,064,256 | 7,005,566 | 6,951,975 | 6,887,708 |
| 6,881,881 | 6,815,585 | 6,759,578 | 6,706,949 | 6,555,732 | 6,333,452 |
| 6,881,880 | 6,815,584 | 6,759,577 | 6,700,041 | 6,555,673 | 6,333,451 |
| 6,878,865 | 6,815,583 | 6,756,530 | 6,693,231 | 6,541,684 | 6,331,661 |
| 6,878,864 | 6,815,578 | 6,756,529 | 6,677,503 | 6,538,177 | 6,329,579 |
| 6,878,863 | 6,812,384 | 6,756,528 | 6,677,502 | 6,538,176 | 6,326,530 |
| 6,875,907 | 6,812,383 | 6,753,464 | 6,667,427 | 6,528,704 | 6,326,529 |
| 6,872,874 | 6,812,380 | 6,750,384 | 6,660,907 | 6,518,487 | 6,323,402 |
| 6,872,873 | 6,809,242 | 6,750,380 | 6,657,107 | 6,518,483 | 6,323,401 |
| 6,864,411 | 6,809,241 | 6,747,196 | 6,646,182 | 6,515,202 | 6,323,400 |
| 6,864,408 | 6,809,236 | 6,747,193 | 6,639,131 | 6,504,084 | 6,323,399 |
| 6,864,407 | 6,806,408 | 6,743,970 | 6,639,126 | 6,504,083 | 6,323,398 |
| 6,861,577 | 6,806,407 | 6,740,798 | 6,635,807 | 6,504,082 | 6,323,015 |
| 6,858,785 | 6,806,406 | 6,740,796 | 6,630,615 | 6,479,730 | 6,320,106 |
| 6,858,782 | 6,806,405 | 6,740,795 | 6,630,614 | 6,476,292 | 6,320,105 |
| 6,858,781 | 6,806,404 | 6,737,566 | 6,627,797 | 6,472,185 | 6,316,704 |
| 6,858,778 | 6,806,401 | 6,737,565 | 6,617,499 | 6,444,874 | 6,316,703 |
| 6,855,878 | 6,803,508 | 6,737,562 | 6,617,498 | 6,441,151 | 6,316,702 |
| 6,855,876 | 6,803,498 | 6,737,560 | 6,613,967 | 6,433,259 | 6,316,700 |
| 6,855,875 | 6,800,796 | 6,734,348 | 6,613,966 | 6,429,362 | 6,313,384 |
| 6,855,874 | 6,800,795 | 6,734,347 | 6,613,965 | 6,426,452 | 6,313,383 |
| 6,855,871 | 6,797,868 | 6,734,345 | 6,613,964 | 6,423,888 | 6,313,382 |
| 6,852,913 | 6,797,867 | 6,734,341 | 6,613,963 | 6,423,886 | 6,313,381 |
| 6,849,791 | 6,797,866 | 6,730,837 | 6,610,911 | 6,410,829 | 6,313,376 |
| 6,849,789 | 6,797,865 | 6,730,836 | 6,610,910 | 6,407,315 | 6,313,375 |
| 6,849,788 | 6,797,864 | 6,730,835 | 6,608,243 | 6,403,862 | 6,310,274 |
| 6,849,787 | 6,797,863 | 6,730,834 | 6,608,240 | 6,403,860 | 6,307,132 |
| 6,846,976 | 6,797,859 | 6,730,829 | 6,605,762 | 6,399,856 | 6,307,131 |
| 6,846,975 | 6,794,563 | 6,727,413 | 6,605,761 | 6,392,127 | 6,303,851 |
| 6,846,974 | 6,791,016 | 6,727,412 | 6,605,760 | 6,392,126 | 6,297,433 |
| 6,844,488 | 6,784,350 | 6,727,410 | 6,605,759 | 6,388,179 | 6,297,432 |
| 6,838,593 | 6,784,349 | 6,723,903 | 6,605,758 | 6,388,171 | 6,297,426 |
| 6,835,877 | 6,784,347 | 6,723,902 | 6,605,757 | 6,388,169 | 6,291,745 |
| 6,835,875 | 6,784,341 | 6,720,487 | 6,605,756 | 6,384,302 | 6,288,310 |
| 6,833,498 | 6,781,043 | 6,720,486 | 6,605,755 | 6,372,961 | 6,287,843 |
| 6,831,215 | 6,781,042 | 6,720,481 | 6,600,095 | 6,369,301 | 6,284,953 |
| 6,828,493 | 6,781,041 | 6,720,478 | 6,586,659 | 6,369,300 | 6,284,950 |
| 6,828,490 | 6,777,599 | 6,720,475 | 6,586,657 | 6,362,400 | 6,284,948 |
| 6,825,405 | 6,777,598 | 6,717,040 | 6,583,343 | 6,359,201 | 6,271,439 |
| 6,825,404 | 6,777,597 | 6,717,039 | 6,583,342 | 6,346,657 | 6,271,437 |
| 6,825,397 | 6,777,596 | 6,717,038 | 6,583,341 | 6,344,603 | 6,268,553 |
| 6,822,144 | 6,777,590 | 6,717,037 | 6,580,018 | 6,342,659 | 6,265,646 |
| 6,822,140 | 6,774,290 | 6,717,036 | 6,576,819 | 6,339,186 | 6,265,636 |
| 6,818,813 | 6,774,289 | 6,717,033 | 6,576,814 | 6,339,144 | 6,259,005 |
| 6,818,811 | 6,774,282 | 6,713,666 | 6,573,433 | 6,337,100 | 6,259,004 |
| 6,818,809 | 6,770,802 | 6,713,665 | 6,566,589 | 6,335,476 | 6,255,090 |
| 6,818,808 | 6,765,132 | 6,706,954 | 6,566,584 | 6,335,197 | 6,248,935 |
| 6,815,592 | 6,759,580 | 6,706,951 | 6,563,020 | 6,333,453 | 6,242,673 |
| 6,242,672 | 6,156,958 | 6,111,167 | 5,990,392 | 5,902,921 | 5,792,909 |
| 6,235,976 | 6,153,817 | 6,107,551 | 5,990,389 | 5,900,526 | 5,792,908 |
| 6,235,972 | 6,153,816 | 6,107,550 | 5,986,185 | 5,900,524 | 5,792,907 |
| 6,232,529 | 6,147,285 | 6,107,545 | 5,986,184 | 5,895,835 | 5,783,190 |
| 6,232,527 | 6,147,284 | 6,100,030 | 5,986,183 | 5,889,188 | 5,773,697 |
| 6,229,079 | 6,147,283 | 6,096,953 | 5,981,854 | 5,866,768 | 5,773,684 |
| 6,229,078 | 6,143,962 | 6,096,951 | 5,977,457 | 5,866,767 | 5,773,682 |
| 6,229,077 | 6,143,956 | 6,096,947 | 5,977,456 | 5,866,766 | 5,770,790 |
| 6,229,074 | 6,143,955 | 6,093,875 | 5,977,451 | 5,866,765 | 5,767,347 |
| 6,229,073 | 6,143,954 | 6,091,007 | 5,977,445 | 5,861,541 | 5,767,344 |
| 6,225,537 | 6,140,562 | 6,091,006 | 5,973,237 | 5,859,354 | 5,767,343 |
| 6,225,529 | 6,140,557 | 6,087,567 | 5,973,234 | 5,859,341 | 5,767,340 |
| 6,222,103 | 6,137,037 | 6,087,566 | 5,962,772 | 5,859,320 | 5,763,757 |
| 6,222,102 | 6,137,036 | 6,087,565 | 5,962,771 | 5,859,319 | 5,763,747 |
| 6,222,101 | 6,133,514 | 6,087,564 | 5,959,185 | 5,859,318 | 5,763,746 |
| 6,215,049 | 6,133,513 | 6,087,559 | 5,955,361 | 5,859,317 | 5,763,744 |
| 6,211,445 | 6,133,508 | 6,084,164 | 5,952,550 | 5,859,316 | 5,763,743 |
| 6,211,440 | 6,130,370 | 6,084,160 | 5,952,549 | 5,859,313 | 5,763,243 |
| 6,211,437 | 6,127,610 | 6,080,919 | 5,948,957 | 5,852,226 | 5,750,868 |
| 6,211,435 | 6,127,609 | 6,080,913 | 5,945,587 | 5,852,225 | 5,750,849 |
| 6,211,434 | 6,127,603 | 6,077,998 | 5,945,586 | 5,850,024 | 5,750,847 |
| 6,211,433 | 6,127,602 | 6,077,997 | 5,942,671 | 5,850,016 | 5,750,843 |
| 6,198,026 | 6,127,600 | 6,077,993 | 5,942,670 | 5,850,013 | 5,750,842 |
| 6,197,561 | 6,124,535 | 6,075,186 | 5,942,669 | 5,850,012 | 5,750,841 |
| 6,194,638 | 6,124,534 | 6,075,182 | 5,942,668 | 5,850,011 | 5,750,839 |
| 6,194,637 | 6,124,533 | 6,072,104 | 5,942,667 | 5,850,010 | 5,750,838 |
| 6,191,343 | 6,124,532 | 6,069,304 | 5,939,608 | 5,850,009 | 5,750,835 |
| 6,188,001 | 6,124,531 | 6,057,491 | 5,939,607 | 5,850,007 | 5,750,834 |
| 6,188,000 | 6,124,530 | 6,054,639 | 5,936,148 | 5,844,118 | 5,750,832 |
| 6,184,448 | 6,124,529 | 6,040,505 | 5,936,147 | 5,844,117 | 5,750,831 |
| 6,184,445 | 6,124,526 | 6,037,530 | 5,936,142 | 5,844,116 | 5,750,829 |
| 6,184,439 | 6,121,524 | 6,037,523 | 5,936,141 | 5,841,015 | 5,741,684 |
| 6,180,857 | 6,121,523 | 6,028,254 | 5,936,140 | 5,827,940 | 5,736,627 |
| 6,180,856 | 6,121,522 | 6,028,252 | 5,929,313 | 5,824,844 | 5,731,499 |
| 6,180,850 | 6,121,520 | 6,025,547 | 5,929,311 | 5,824,524 | 5,731,497 |
| 6,177,613 | 6,121,514 | 6,020,543 | 5,929,301 | 5,817,918 | 5,731,496 |
| 6,177,611 | 6,118,056 | 6,018,113 | 5,920,002 | 5,811,651 | 5,731,494 |
| 6,175,065 | 6,118,055 | 6,018,112 | 5,917,134 | 5,811,650 | 5,731,493 |
| 6,175,058 | 6,118,054 | 6,018,111 | 5,917,130 | 5,811,639 | 5,731,492 |
| 6,169,234 | 6,118,053 | 6,018,110 | 5,917,129 | 5,811,638 | 5,731,491 |
| 6,166,305 | 6,118,052 | 6,018,109 | 5,917,125 | 5,811,637 | 5,728,926 |
| 6,166,303 | 6,118,051 | 6,015,941 | 5,912,417 | 5,804,692 | 5,728,921 |
| 6,166,296 | 6,114,614 | 6,013,859 | 5,910,634 | 5,804,691 | 5,728,920 |
| 6,162,968 | 6,114,613 | 6,005,172 | 5,910,633 | 5,792,915 | 5,728,919 |
| 6,162,964 | 6,114,612 | 6,005,168 | 5,908,976 | 5,792,912 | 5,728,558 |
| 6,160,211 | 6,114,607 | 5,998,711 | 5,907,086 | 5,792,911 | 5,723,723 |
| 6,160,209 | 6,111,173 | 5,990,393 | 5,905,189 | 5,792,910 | 5,723,722 |

| | | | | |
|---|---|---|---|---|
| 5,717,129 | 5,541,352 | 5,159,133 | 6,495,738 | 5,451,514 |
| 5,708,189 | 5,534,661 | 5,159,132 | 6,410,828 | |
| 5,689,036 | 5,530,184 | 5,157,208 | 6,384,207 | |
| 5,689,034 | 5,527,986 | 5,157,206 | 6,331,664 | |
| 5,675,066 | 5,506,368 | 5,097,096 | 6,323,395 | |
| 5,639,946 | 5,506,367 | 5,097,095 | 6,166,302 | |
| 5,638,637 | 5,502,272 | 5,097,093 | 6,048,838 | |
| 5,633,427 | 5,495,069 | 5,097,092 | 7,799,906 | |
| 5,625,133 | 5,495,066 | 5,095,174 | 7,723,584 | |
| 5,625,132 | 5,495,065 | 5,082,992 | 7,709,697 | |
| 5,625,130 | 5,491,295 | 5,082,991 | 7,674,951 | |
| 5,625,129 | 5,491,290 | 5,049,503 | 7,663,023 | |
| 5,618,987 | 5,491,289 | 4,996,049 | 7,598,430 | |
| 5,608,140 | 5,491,287 | 4,812,600 | 7,288,409 | |
| 5,608,139 | 5,491,286 | 4,812,599 | 7,232,941 | |
| 5,608,138 | 5,478,369 | 4,806,669 | 7,148,406 | |
| 5,602,318 | 5,476,999 | 4,806,652 | 7,135,616 | |
| 5,602,317 | 5,463,173 | 4,737,596 | 7,087,426 | |
| 5,602,312 | 5,461,171 | 4,731,499 | 7,071,384 | |
| 5,585,538 | 5,453,564 | 7,820,883 | 7,071,376 | |
| 5,585,537 | 5,444,178 | 7,795,395 | 7,049,485 | |
| 5,583,210 | 5,436,390 | 7,728,195 | 7,012,172 | |
| 5,576,472 | 5,434,346 | 7,723,582 | 6,906,239 | |
| 5,574,209 | 5,432,068 | 7,723,581 | 6,855,864 | |
| 5,574,208 | 5,426,041 | 7,723,580 | 6,831,208 | |
| 5,569,822 | 5,416,254 | 7,723,579 | 6,703,539 | |
| 5,569,821 | 5,387,758 | 7,723,578 | 6,653,528 | |
| 5,569,819 | 5,387,755 | 7,723,577 | 6,635,805 | |
| 5,569,818 | 5,387,754 | 7,718,852 | 6,610,908 | |
| 5,569,817 | 5,367,109 | 7,709,707 | 6,489,538 | |
| 5,569,816 | 5,365,014 | 7,709,623 | 6,479,732 | |
| 5,567,861 | 5,356,799 | 7,671,253 | 6,476,291 | |
| 5,563,326 | 5,354,941 | 7,667,100 | 6,455,762 | |
| 5,563,325 | 5,349,119 | 7,635,798 | 6,441,272 | |
| 5,563,323 | 5,347,081 | 7,566,818 | 6,252,135 | |
| 5,563,322 | 5,347,080 | 7,456,340 | 6,242,381 | |
| 5,563,321 | 5,347,079 | 7,411,117 | 6,211,432 | |
| 5,563,320 | 5,316,930 | 7,371,935 | 6,204,434 | |
| 5,563,055 | 5,304,720 | 7,355,100 | 6,066,780 | |
| 5,557,038 | 5,304,719 | 7,348,473 | 6,015,943 | |
| 5,557,035 | 5,285,004 | 7,332,657 | 5,981,837 | |
| 5,557,034 | 5,276,265 | 7,179,965 | 5,959,178 | |
| 5,545,814 | 5,276,264 | 7,071,386 | 5,952,486 | |
| 5,545,813 | 5,260,503 | 7,071,385 | 5,922,928 | |
| 5,545,812 | 5,245,125 | 6,791,013 | 5,886,243 | |
| 5,545,809 | 5,220,114 | 6,753,460 | 5,850,020 | |
| 5,543,575 | 5,159,134 | 6,753,459 | 5,646,333 | |

| | | |
|---|---|---|
| 20100071092 | 20100293639 | 20100275304 |
| 20100058498 | 20100293638 | 20100275303 |
| 20100058496 | 20100293637 | 20100275301 |
| 20090119796 | 20100293634 | 20100275300 |
| 20090106862 | 20100293633 | 20100275299 |
| 20090044294 | 20100293632 | 20100275298 |
| 20090019605 | 20100293630 | 20100275297 |
| 20080313774 | 20100293629 | 20100275296 |
| 20080235820 | 20100287653 | 20100275295 |
| 20080213871 | 20100287652 | 20100275294 |
| 20080118954 | 20100287651 | 20100275293 |
| 20080058510 | 20100287650 | 20100275292 |
| 20060206964 | 20100287649 | 20100275291 |
| 20060130183 | 20100287648 | 20100275290 |
| 20060101535 | 20100287647 | 20100275289 |
| 20050091707 | 20100287646 | 20100275288 |
| 20050076403 | 20100287645 | 20100275287 |
| 20040049802 | 20100287644 | 20100275286 |
| 20030163839 | 20100287643 | 20100273987 |
| 20030131373 | 20100287642 | 20100272880 |
| 20020138870 | 20100285202 | 20100269224 |
| 20020078477 | 20100281564 | 20100269211 |
| 20020078474 | 20100281563 | 20100269210 |
| 20100293661 | 20100281562 | 20100269209 |
| 20100293660 | 20100281561 | 20100269208 |
| 20100293659 | 20100281560 | 20100269207 |
| 20100293658 | 20100281559 | 20100269206 |
| 20100293657 | 20100281558 | 20100269205 |
| 20100293656 | 20100281557 | 20100269204 |
| 20100293655 | 20100278996 | 20100269203 |
| 20100293654 | 20100275332 | 20100269202 |
| 20100293653 | 20100275322 | 20100269201 |
| 20100293652 | 20100275318 | 20100269200 |
| 20100293651 | 20100275317 | 20100269199 |
| 20100293650 | 20100275316 | 20100269198 |
| 20100293649 | 20100275315 | 20100269197 |
| 20100293648 | 20100275314 | 20100269196 |
| 20100293647 | 20100275313 | 20100269195 |
| 20100293646 | 20100275312 | 20100263083 |
| 20100293645 | 20100275310 | 20100263082 |
| 20100293644 | 20100275309 | 20100263081 |
| 20100293643 | 20100275308 | 20100263080 |
| 20100293642 | 20100275307 | 20100263079 |
| 20100293640 | 20100275305 | 20100263078 |
| 20100263077 | 20100115649 | 20090288198 |
| 20100263076 | 20100115648 | 20090288197 |
| 20100263075 | 20100115647 | 20090288195 |
| 20100263074 | 20100115646 | 20090288194 |
| 20100263073 | 20100115645 | 20090288191 |
| 20100263072 | 20100115644 | 20090288189 |
| 20100263071 | 20100115643 | 20090288188 |
| 20100263070 | 20100112182 | 20090282575 |
| 20100263069 | 20100107272 | 20090282574 |
| 20100263068 | 20100107271 | 20090282573 |
| 20100260921 | 20100107270 | 20090282572 |
| 20100260920 | 20100107268 | 20090282571 |
| 20100257630 | 20100107267 | 20090282570 |
| 20100257629 | 20100100980 | 20090282569 |
| 20100257628 | 20100080887 | 20090282568 |
| 20100257627 | 20100064394 | 20090282567 |
| 20100257626 | 20100043094 | 20090282566 |
| 20100257625 | 20100043093 | 20090282565 |
| 20100257621 | 20100043091 | 20090282564 |
| 20100255175 | 20100043090 | 20090282563 |
| 20100251412 | 20100043088 | 20090282562 |
| 20100251411 | 20100043087 | 20090282561 |
| 20100251410 | 20100043086 | 20090282560 |
| 20100251408 | 20100037339 | 20090282559 |
| 20100251407 | 20100037338 | 20090282558 |
| 20100251406 | 20100037337 | 20090282557 |
| 20100251405 | 20100037336 | 20090282556 |
| 20100251403 | 20100037333 | 20090282555 |
| 20100251402 | 20100024064 | 20090282554 |
| 20100251401 | 20100024063 | 20090282553 |
| 20100251400 | 20100024062 | 20090282552 |
| 20100251399 | 20100024054 | 20090282551 |
| 20100251398 | 20100024052 | 20090282550 |
| 20100249389 | 20090288216 | 20090282549 |
| 20100248963 | 20090288215 | 20090282548 |
| 20100247733 | 20090288213 | 20090282547 |
| 20100242132 | 20090288212 | 20090282546 |
| 20100242130 | 20090288211 | 20090282545 |
| 20100242129 | 20090288210 | 20090282544 |
| 20100218269 | 20090288208 | 20090282543 |
| 20100196580 | 20090288207 | 20090282542 |
| 20100192245 | 20090288206 | 20090282541 |
| 20100173061 | 20090288205 | 20090282540 |
| 20100168455 | 20090288203 | 20090282539 |
| 20100146656 | 20090288202 | 20090282538 |
| 20100138953 | 20090288201 | 20090282536 |
| 20100115652 | 20090288200 | 20090282535 |
| 20090282534 | 20090276895 | 20090081353 |
| 20090282533 | 20090276894 | 20090077694 |
| 20090282532 | 20090276893 | 20090070902 |
| 20090282531 | 20090276892 | 20090070891 |
| 20090282530 | 20090276891 | 20090055957 |
| 20090282529 | 20090276885 | 20090055956 |
| 20090282528 | 20090276884 | 20090055955 |
| 20090282527 | 20090276883 | 20090031438 |
| 20090282526 | 20090276882 | 20090029861 |
| 20090282525 | 20090276881 | 20090019604 |
| 20090282523 | 20090276880 | 20090019603 |
| 20090282522 | 20090276879 | 20090019595 |
| 20090282521 | 20090276878 | 20090019594 |
| 20090282520 | 20090276871 | 20090019593 |
| 20090282519 | 20090276870 | 20090019592 |
| 20090282517 | 20090276869 | 20090019591 |
| 20090282516 | 20090275741 | 20090019590 |
| 20090282515 | 20090246350 | 20090019589 |
| 20090282514 | 20090241213 | 20090019588 |
| 20090282513 | 20090241212 | 20090019587 |
| 20090282512 | 20090241211 | 20090019586 |
| 20090282511 | 20090241210 | 20090019585 |
| 20090282510 | 20090235379 | 20090019584 |
| 20090282509 | 20090235378 | 20090019583 |
| 20090282508 | 20090235377 | 20090019582 |
| 20090282500 | 20090229004 | 20090019581 |
| 20090282499 | 20090229003 | 20090019580 |
| 20090282498 | 20090229002 | 20090019579 |
| 20090276916 | 20090210961 | 20090019578 |
| 20090276915 | 20090169709 | 20090019577 |
| 20090276914 | 20090165163 | 20090019576 |
| 20090276913 | 20090165162 | 20090019575 |
| 20090276910 | 20090165161 | 20090019574 |
| 20090276909 | 20090165159 | 20090019573 |
| 20090276908 | 20090165158 | 20090019572 |
| 20090276907 | 20090151020 | 20090019571 |
| 20090276906 | 20090138989 | 20090019570 |
| 20090276905 | 20090138985 | 20090019569 |
| 20090276904 | 20090133147 | 20090019568 |
| 20090276903 | 20090133146 | 20090019567 |
| 20090276902 | 20090133145 | 20090019565 |
| 20090276901 | 20090133144 | 20090019564 |
| 20090276900 | 20090133143 | 20090013429 |
| 20090276899 | 20090133142 | 20090013428 |
| 20090276898 | 20090100536 | 20090013427 |
| 20090276897 | 20090098099 | 20090013426 |
| 20090276896 | 20090083886 | 20090013425 |
| 20090007290 | 20080313765 | 20080313760 |
| 20090007289 | 20080313764 | 20080313759 |
| 20090007288 | 20080313763 | 20080313758 |
| 20090007287 | 20080313762 | 20080313757 |
| 20090007286 | 20080313761 | 20080282432 |
| 20080282422 | 20080282378 | 20080263712 |
| 20080282421 | 20080282377 | 20080263711 |
| 20080282420 | 20080282376 | 20080263706 |
| 20080282419 | 20080282375 | 20080263705 |
| 20080282418 | 20080282374 | 20080260929 |
| 20080282417 | 20080282373 | 20080256669 |
| 20080282416 | 20080282372 | 20080235819 |
| 20080282415 | 20080282371 | 20080227639 |
| 20080282414 | 20080282370 | 20080216190 |
| 20080282413 | 20080282366 | 20080216189 |
| 20080282412 | 20080280361 | 20080178345 |
| 20080282411 | 20080276330 | 20080178344 |
| 20080282410 | 20080271197 | 20080178343 |
| 20080282409 | 20080271196 | 20080178342 |
| 20080282408 | 20080271195 | 20080178341 |
| 20080282407 | 20080271194 | 20080178340 |
| 20080282406 | 20080271193 | 20080178338 |
| 20080282405 | 20080271192 | 20080178337 |
| 20080282404 | 20080271191 | 20080178336 |
| 20080282403 | 20080271190 | 20080178335 |
| 20080282402 | 20080271189 | 20080178334 |
| 20080282401 | 20080271188 | 20080178333 |
| 20080282400 | 20080271187 | 20080178332 |
| 20080282399 | 20080271186 | 20080178331 |
| 20080282398 | 20080271185 | 20080178330 |
| 20080282397 | 20080271184 | 20080178329 |
| 20080282396 | 20080271183 | 20080178328 |
| 20080282395 | 20080271182 | 20080178327 |
| 20080282394 | 20080271181 | 20080178326 |
| 20080282393 | 20080271180 | 20080178322 |
| 20080282392 | 20080271179 | 20080178320 |
| 20080282389 | 20080271178 | 20080178319 |
| 20080282388 | 20080271177 | 20080178318 |
| 20080282387 | 20080271176 | 20080178317 |
| 20080282386 | 20080271175 | 20080172761 |
| 20080282385 | 20080271174 | 20080172756 |
| 20080282384 | 20080271173 | 20080172755 |
| 20080282383 | 20080271172 | 20080172754 |
| 20080282382 | 20080271171 | 20080168576 |
| 20080282381 | 20080271170 | 20080155711 |
| 20080282380 | 20080271168 | 20080155710 |
| 20080282379 | 20080263713 | 20080155708 |
| 20080155707 | 20060162007 | 20040237150 |
| 20080148428 | 20060111254 | 20040237149 |
| 20080148427 | 20060107348 | 20040237148 |
| 20080148426 | 20060101543 | 20040237139 |
| 20080148425 | 20060070140 | 20040221346 |
| 20080148424 | 20060064777 | 20040221344 |
| 20080148423 | 20060064776 | 20040221343 |
| 20080148422 | 20060059590 | 20040221342 |
| 20080148421 | 20060059589 | 20040221341 |
| 20080148420 | 20060021081 | 20040221339 |
| 20080070296 | 20060010530 | 20040221335 |
| 20080066202 | 20060010529 | 20040221329 |
| 20080064866 | 20060010528 | 20040221328 |
| 20080050506 | 20060010527 | 20040210958 |
| 20080022423 | 20060010526 | 20040205862 |
| 20070266456 | 20060010525 | 20040205861 |
| 20070256190 | 20060010524 | 20040205860 |
| 20070256187 | 20060010523 | 20040205859 |
| 20070256186 | 20060010522 | 20040205857 |
| 20070256185 | 20060010521 | 20040205856 |
| 20070256184 | 20050289664 | 20040205854 |
| 20070256182 | 20050193440 | 20040205849 |
| 20070256181 | 20050193438 | 20040168228 |
| 20070256180 | 20050193437 | 20040168225 |
| 20070256179 | 20050193436 | 20040168224 |
| 20070256171 | 20050183155 | 20040168223 |
| 20070256170 | 20050183154 | 20040168222 |
| 20070256155 | 20050183153 | 20040168219 |
| 20070250957 | 20050155114 | 20040148665 |
| 20070250955 | 20050155106 | 20040148664 |
| 20070250954 | 20050144680 | 20040148663 |
| 20070250952 | 20050144679 | 20040148662 |
| 20070250951 | 20050144678 | 20040148660 |
| 20070250950 | 20050144677 | 20040148659 |
| 20070250949 | 20050144676 | 20040148650 |
| 20070250947 | 20050144675 | 20040132975 |
| 20070214516 | 20050144674 | 20040111772 |
| 20070067871 | 20050132437 | 20040111770 |
| 20070054400 | 20050114929 | 20040093637 |
| 20070037708 | 20050114928 | 20040060082 |
| 20070022494 | 20050079494 | 20040055059 |
| 20070011761 | 20050071900 | 20040055058 |
| 20060288451 | 20050022272 | 20040055057 |
| 20060288447 | 20050022261 | 20040055056 |
| 20060282915 | 20050005332 | 20040055055 |
| 20060265778 | 20050005321 | 20040055054 |
| 20060174372 | 20040237152 | 20040055051 |
| 20040055049 | 20100175149 | 20090089891 |
| 20040055048 | 20100162434 | 20090083882 |
| 20040055047 | 20100162432 | 20090075819 |
| 20040055045 | 20100162431 | 20090064374 |
| 20040055044 | 20100162430 | 20090055970 |
| 20040055043 | 20100162429 | 20090038025 |
| 20040049821 | 20100132071 | 20090031451 |
| 20040049820 | 20100115664 | 20090031446 |
| 20040049817 | 20100095404 | 20090031440 |
| 20040049816 | 20100095403 | 20090029860 |
| 20040049815 | 20100093715 | 20080307543 |
| 20040049814 | 20100088784 | 20080301835 |
| 20040016030 | 20100088783 | 20080263725 |
| 20040010824 | 20100088782 | 20080229453 |
| 20040010823 | 20100071093 | 20080229452 |
| 20030237111 | 20100071091 | 20080209588 |
| 20030233679 | 20100043100 | 20080201799 |
| 20030232757 | 20100037350 | 20080189806 |
| 20030195336 | 20100031392 | 20080178356 |
| 20030192072 | 20100011466 | 20080178355 |
| 20030182682 | 20100011465 | 20080172759 |
| 20030182678 | 20100005542 | 20080168585 |
| 20030172416 | 20090320158 | 20080168578 |
| 20030154524 | 20090320157 | 20080168577 |
| 20030131375 | 20090282580 | 20080163398 |
| 20030119158 | 20090276921 | 20080127369 |
| 20030101482 | 20090249514 | 20080120748 |
| 20030097672 | 20090241230 | 20080076179 |
| 20030068335 | 20090241227 | 20080072350 |
| 20030056243 | 20090235389 | 20080072347 |
| 20030005491 | 20090217417 | 20080052794 |
| 20020152496 | 20090217406 | 20080052792 |
| 20100293665 | 20090203094 | 20080050820 |
| 20100287665 | 20090188004 | 20080022427 |
| 20100287641 | 20090186762 | 20080005808 |
| 20100269219 | 20090172834 | 20070294783 |
| 20100263088 | 20090170173 | 20070261132 |
| 20100251416 | 20090165173 | 20070226842 |
| 20100229259 | 20090165170 | 20070209092 |
| 20100227924 | 20090158461 | 20070209087 |
| 20100205690 | 20090158454 | 20070199103 |
| 20100205689 | 20090144859 | 20070174927 |
| 20100199382 | 20090138987 | 20070118920 |
| 20100199380 | 20090113572 | 20070111311 |
| 20100199379 | 20090100541 | 20070033670 |
| 20100192254 | 20090100540 | 20070022497 |
| 20100190794 | 20090089896 | 20060206961 |
| 20060179515 | 20100192253 | 20090288217 |
| 20060174373 | 20100190707 | 20090282586 |
| 20060168684 | 20100186116 | 20090275473 |
| 20060162021 | 20100186115 | 20090265818 |
| 20060137043 | 20100186113 | 20090265802 |
| 20060112452 | 20100175150 | 20090264351 |
| 20060064784 | 20100167403 | 20090264290 |
| 20060037102 | 20100162440 | 20090260106 |
| 20060010514 | 20100162425 | 20090260105 |
| 20050241020 | 20100162424 | 20090235392 |
| 20040194163 | 20100154083 | 20090229018 |
| 20100293670 | 20100132072 | 20090227013 |
| 20100287669 | 20100132070 | 20090222954 |
| 20100287662 | 20100122367 | 20090222943 |
| 20100287656 | 20100115667 | 20090210970 |
| 20100287655 | 20100115662 | 20090205078 |
| 20100285591 | 20100115661 | 20090205067 |
| 20100281579 | 20100100985 | 20090205065 |
| 20100281578 | 20100100981 | 20090192117 |
| 20100281570 | 20100095401 | 20090192116 |
| 20100281569 | 20100088785 | 20090188008 |
| 20100269229 | 20100077508 | 20090188003 |
| 20100269228 | 20100077507 | 20090183285 |
| 20100269221 | 20100071087 | 20090183279 |
| 20100269218 | 20100058495 | 20090183278 |
| 20100269194 | 20100050293 | 20090183277 |
| 20100269193 | 20100037347 | 20090183276 |
| 20100269189 | 20100031391 | 20090178159 |
| 20100269188 | 20100029725 | 20090172841 |
| 20100269187 | 20100017915 | 20090165177 |
| 20100269186 | 20100017908 | 20090165174 |
| 20100269185 | 20100017907 | 20090158471 |
| 20100242138 | 20100017906 | 20090158459 |
| 20100242137 | 20100017905 | 20090158458 |
| 20100242131 | 20090328252 | 20090151022 |
| 20100236146 | 20090325804 | 20090151017 |
| 20100235944 | 20090320163 | 20090144843 |
| 20100235939 | 20090320162 | 20090138986 |
| 20100229257 | 20090320156 | 20090137395 |
| 20100223695 | 20090320155 | 20090136646 |
| 20100221238 | 20090313722 | 20090133161 |
| 20100218276 | 20090313721 | 20090119804 |
| 20100212049 | 20090307800 | 20090119799 |
| 20100199383 | 20090307797 | 20090113570 |
| 20100192263 | 20090300789 | 20090106860 |
| 20100192256 | 20090293147 | 20090106859 |
| 20100192255 | 20090293141 | 20090106857 |
| 20090094713 | 20080256666 | 20080078004 |
| 20090094712 | 20080244767 | 20080072346 |
| 20090089897 | 20080244766 | 20080072345 |
| 20090089895 | 20080244765 | 20080072344 |
| 20090081354 | 20080241927 | 20080072343 |
| 20090077691 | 20080235827 | 20080060099 |
| 20090077690 | 20080234130 | 20080057564 |
| 20090077689 | 20080227091 | 20080051288 |
| 20090077688 | 20080222753 | 20080047031 |
| 20090069182 | 20080216200 | 20080040825 |
| 20090055966 | 20080216191 | 20080040824 |
| 20090055961 | 20080209582 | 20080034652 |
| 20090049571 | 20080200415 | 20080034448 |
| 20090049570 | 20080189810 | 20080034447 |
| 20090049569 | 20080178353 | 20080022426 |
| 20090038034 | 20080178350 | 20080020968 |
| 20090038028 | 20080178325 | 20080020967 |
| 20090038027 | 20080178323 | 20080020966 |
| 20090038026 | 20080172762 | 20080020123 |
| 20090036308 | 20080171321 | 20080016596 |
| 20090035765 | 20080168581 | 20080016595 |
| 20090031449 | 20080163402 | 20080016594 |
| 20090031448 | 20080163401 | 20070300323 |
| 20090025103 | 20080163400 | 20070294781 |
| 20090025102 | 20080141392 | 20070289030 |
| 20090007302 | 20080134362 | 20070283461 |
| 20090005306 | 20080134361 | 20070277256 |
| 20090000188 | 20080127377 | 20070274972 |
| 20090000187 | 20080127375 | 20070271628 |
| 20080320617 | 20080124804 | 20070266462 |
| 20080320613 | 20080124797 | 20070266458 |
| 20080313777 | 20080124796 | 20070261136 |
| 20080313770 | 20080120747 | 20070256198 |
| 20080305238 | 20080120746 | 20070250959 |
| 20080299658 | 20080120745 | 20070245430 |
| 20080286434 | 20080120744 | 20070245429 |
| 20080280018 | 20080120743 | 20070245428 |
| 20080280017 | 20080120742 | 20070245427 |
| 20080280016 | 20080120741 | 20070245425 |
| 20080280015 | 20080115241 | 20070240238 |
| 20080280014 | 20080109925 | 20070238179 |
| 20080280013 | 20080109924 | 20070234444 |
| 20080280012 | 20080108072 | 20070234443 |
| 20080280011 | 20080090294 | 20070231905 |
| 20080274261 | 20080090293 | 20070226837 |
| 20080274260 | 20080086783 | 20070226836 |
| 20080271198 | 20080083043 | 20070226835 |
| 20070226834 | 20070136866 | 20070107091 |
| 20070226833 | 20070136865 | 20070107090 |
| 20070226832 | 20070136864 | 20070107089 |
| 20070226831 | 20070136863 | 20070107088 |
| 20070220627 | 20070136862 | 20070107084 |
| 20070220626 | 20070136861 | 20070094747 |
| 20070214514 | 20070136860 | 20070089200 |
| 20070199105 | 20070136859 | 20070089181 |
| 20070192899 | 20070136858 | 20070079402 |
| 20070192897 | 20070136857 | 20070079397 |
| 20070180578 | 20070136856 | 20070079393 |
| 20070180577 | 20070136855 | 20070074311 |
| 20070180576 | 20070136854 | 20070074303 |
| 20070169227 | 20070136853 | 20070061926 |
| 20070169226 | 20070136852 | 20070044180 |
| 20070169225 | 20070136851 | 20070016980 |
| 20070169220 | 20070136850 | 20070011771 |
| 20070162999 | 20070136849 | 20070006350 |
| 20070157342 | 20070136848 | 20060294625 |
| 20070157341 | 20070136847 | 20060294624 |
| 20070157335 | 20070136846 | 20060293913 |
| 20070150980 | 20070136845 | 20060288453 |
| 20070150979 | 20070136844 | 20060288448 |
| 20070143880 | 20070136843 | 20060288440 |
| 20070136891 | 20070136842 | 20060282918 |
| 20070136888 | 20070136838 | 20060281910 |
| 20070136887 | 20070130653 | 20060272058 |
| 20070136886 | 20070130652 | 20060272057 |
| 20070136885 | 20070130651 | 20060272055 |
| 20070136884 | 20070130650 | 20060272054 |
| 20070136883 | 20070130649 | 20060272046 |
| 20070136882 | 20070130648 | 20060260006 |
| 20070136881 | 20070130647 | 20060242733 |
| 20070136880 | 20070130646 | 20060225161 |
| 20070136879 | 20070130642 | 20060225160 |
| 20070136878 | 20070118919 | 20060225152 |
| 20070136877 | 20070107102 | 20060225151 |
| 20070136876 | 20070107101 | 20060223102 |
| 20070136875 | 20070107100 | 20060212971 |
| 20070136874 | 20070107099 | 20060212966 |
| 20070136873 | 20070107098 | 20060212964 |
| 20070136872 | 20070107097 | 20060206967 |
| 20070136871 | 20070107096 | 20060200874 |
| 20070136870 | 20070107095 | 20060195954 |
| 20070136869 | 20070107094 | 20060195953 |
| 20070136868 | 20070107093 | 20060195937 |
| 20070136867 | 20070107092 | 20060185039 |
| 20060174382 | 20060107413 | 20060107365 |
| 20060174381 | 20060107412 | 20060107364 |
| 20060168692 | 20060107411 | 20060107363 |
| 20060162030 | 20060107410 | 20060107362 |
| 20060162027 | 20060107409 | 20060107361 |
| 20060162015 | 20060107408 | 20060107360 |
| 20060162009 | 20060107407 | 20060107359 |
| 20060156439 | 20060107406 | 20060107358 |
| 20060150277 | 20060107405 | 20060107357 |
| 20060150275 | 20060107404 | 20060107356 |
| 20060150274 | 20060107403 | 20060107355 |
| 20060143744 | 20060107402 | 20060107354 |
| 20060143743 | 20060107401 | 20060107347 |
| 20060143733 | 20060107400 | 20060101546 |
| 20060143728 | 20060107399 | 20060095991 |
| 20060137035 | 20060107398 | 20060095990 |
| 20060137033 | 20060107397 | 20060090225 |
| 20060130190 | 20060107396 | 20060070143 |
| 20060130189 | 20060107395 | 20060070139 |
| 20060130188 | 20060107394 | 20060064789 |
| 20060130187 | 20060107393 | 20060064786 |
| 20060130184 | 20060107391 | 20060064779 |
| 20060112465 | 20060107390 | 20060064773 |
| 20060112464 | 20060107389 | 20060037109 |
| 20060112463 | 20060107388 | 20060037095 |
| 20060112462 | 20060107387 | 20060031962 |
| 20060112461 | 20060107386 | 20060026710 |
| 20060112460 | 20060107385 | 20060026709 |
| 20060112459 | 20060107384 | 20060026708 |
| 20060112458 | 20060107383 | 20060026707 |
| 20060112457 | 20060107382 | 20060021082 |
| 20060112456 | 20060107381 | 20060015968 |
| 20060112444 | 20060107380 | 20060010534 |
| 20060107427 | 20060107379 | 20060005287 |
| 20060107426 | 20060107378 | 20060005275 |
| 20060107425 | 20060107377 | 20060005274 |
| 20060107424 | 20060107376 | 20060005269 |
| 20060107423 | 20060107375 | 20050289670 |
| 20060107422 | 20060107374 | 20050283862 |
| 20060107421 | 20060107373 | 20050283314 |
| 20060107420 | 20060107372 | 20050278812 |
| 20060107419 | 20060107371 | 20050278811 |
| 20060107418 | 20060107370 | 20050278810 |
| 20060107417 | 20060107369 | 20050278805 |
| 20060107416 | 20060107368 | 20050278803 |
| 20060107415 | 20060107367 | 20050273889 |
| 20060107414 | 20060107366 | 20050273881 |
| 20050273879 | 20050150020 | 20050120404 |
| 20050257298 | 20050150019 | 20050114955 |
| 20050257289 | 20050150018 | 20050114954 |
| 20050246798 | 20050150017 | 20050114953 |
| 20050246796 | 20050144690 | 20050114952 |
| 20050235383 | 20050144689 | 20050114951 |
| 20050229483 | 20050144688 | 20050114950 |
| 20050229274 | 20050144687 | 20050114949 |
| 20050229271 | 20050144686 | 20050114948 |
| 20050223443 | 20050144685 | 20050114947 |
| 20050223439 | 20050144683 | 20050114946 |
| 20050223432 | 20050144682 | 20050114945 |
| 20050223426 | 20050144681 | 20050114944 |
| 20050216977 | 20050138697 | 20050114943 |
| 20050216974 | 20050138696 | 20050114942 |
| 20050216968 | 20050138695 | 20050114941 |
| 20050210551 | 20050138686 | 20050114940 |
| 20050210550 | 20050132453 | 20050114939 |
| 20050204418 | 20050132452 | 20050114938 |
| 20050202486 | 20050132451 | 20050114937 |
| 20050198706 | 20050132450 | 20050114936 |
| 20050198702 | 20050125866 | 20050114935 |
| 20050188441 | 20050120443 | 20050114934 |
| 20050188440 | 20050120442 | 20050114933 |
| 20050188439 | 20050120441 | 20050114932 |
| 20050188437 | 20050120440 | 20050114931 |
| 20050177898 | 20050120439 | 20050114930 |
| 20050177897 | 20050120438 | 20050108796 |
| 20050177892 | 20050120437 | 20050108795 |
| 20050172369 | 20050120436 | 20050108794 |
| 20050172368 | 20050120435 | 20050102717 |
| 20050172367 | 20050120434 | 20050097636 |
| 20050166291 | 20050120433 | 20050097634 |
| 20050166287 | 20050120432 | 20050097633 |
| 20050166286 | 20050120431 | 20050081265 |
| 20050166284 | 20050120430 | 20050076404 |
| 20050160506 | 20050120429 | 20050071901 |
| 20050160505 | 20050120428 | 20050070697 |
| 20050160496 | 20050120427 | 20050050590 |
| 20050160494 | 20050120426 | 20050039226 |
| 20050160488 | 20050120425 | 20050034193 |
| 20050155118 | 20050120424 | 20050028231 |
| 20050155102 | 20050120423 | 20050010975 |
| 20050150025 | 20050120422 | 20040248304 |
| 20050150023 | 20050120421 | 20040237147 |
| 20050150022 | 20050120420 | 20040231017 |
| 20050150021 | 20050120419 | 20040216192 |
| 20040210963 | 20040172707 | 20030200557 |
| 20040210960 | 20040172706 | 20030167532 |
| 20040210043 | 20040172705 | 20030167530 |
| 20040205864 | 20040172704 | 20030167528 |
| 20040205863 | 20040172703 | 20030167506 |
| 20040199965 | 20040172702 | 20030167504 |
| 20040199960 | 20040172701 | 20030166855 |
| 20040199959 | 20040172700 | 20030163840 |
| 20040194171 | 20040172699 | 20030163839 |
| 20040194170 | 20040172698 | 20030163838 |
| 20040194169 | 20040172697 | 20030159185 |
| 20040194168 | 20040172696 | 20030150016 |
| 20040194167 | 20040172695 | 20030150014 |
| 20040187179 | 20040172694 | 20030140381 |
| 20040181836 | 20040172693 | 20030140369 |
| 20040181835 | 20040172692 | 20030140368 |
| 20040181833 | 20040172691 | 20030131381 |
| 20040181832 | 20040172668 | 20030110528 |
| 20040181824 | 20040168212 | 20030101484 |
| 20040180436 | 20040166563 | 20030101483 |
| 20040177420 | 20040154059 | 20030097689 |
| 20040177419 | 20040148654 | 20030097680 |
| 20040177418 | 20040148652 | 20030088890 |
| 20040177417 | 20040143871 | 20030084486 |
| 20040177416 | 20040143870 | 20030084476 |
| 20040177415 | 20040123345 | 20030079251 |
| 20040177414 | 20040122592 | 20030079247 |
| 20040177413 | 20040118754 | 20030073239 |
| 20040177412 | 20040111761 | 20030041348 |
| 20040177411 | 20040098769 | 20030033632 |
| 20040177410 | 20040098760 | 20030033629 |
| 20040177409 | 20040083500 | 20030028921 |
| 20040177408 | 20040082770 | 20030028920 |
| 20040177407 | 20040073971 | 20030017566 |
| 20040177406 | 20040068767 | 20030009782 |
| 20040172728 | 20040064856 | 20030005482 |
| 20040172727 | 20040049804 | 20020166146 |
| 20040172726 | 20040040056 | 20020166143 |
| 20040172725 | 20040025206 | 20020166141 |
| 20040172724 | 20040019931 | 20020162142 |
| 20040172723 | 20040005713 | 20020148007 |
| 20040172722 | 20030226178 | 20020144307 |
| 20040172721 | 20030226167 | 20020124284 |
| 20040172711 | 20030221224 | 20020108149 |
| 20040172710 | 20030217387 | 20020083493 |
| 20040172709 | 20030213016 | 20020078475 |
| 20040172708 | 20030213014 | 20020069428 |
| 20020029392 | 20100004177 | 20080085856 |
| 20020004940 | 20090320159 | 20080058248 |
| 20010023501 | 20090203612 | 20070220629 |
| 20100281574 | 20090158473 | 20070143876 |
| 20100257638 | 20090093366 | 20060168683 |
| 20100235943 | 20090029863 | 20060070139 |
| 20100169999 | 20090023782 | 20060037095 |
| 20100168452 | 20080295195 | 20050216969 |
| 20100041610 | 20080260933 | 20050039226 |
| 20100037358 | 20080178323 | 20040194164 |

What is claimed is:

1. A method of making a product, the method comprising:
treating with electron beam irradiation a lignocellulosic feedstock obtained from a plant that has been genetically modified to enhance the production of one or more amino acids, and
saccharifying the treated lignocellulosic feedstock; and
fermenting the saccharified feedstock with an organism
wherein the fermentation is enhanced due to the amino acid present in the genetically modified lignocellulosic feedstock.

2. The method of claim 1, wherein the product is an organic acid.

3. The method of claim 4, wherein the organic acid is selected from the group consisting of lactic acid, propionic acid, butyric acid, succinic acid, 3-hydroxypropionic acid, a salt of any of the acids, an ester of any of the acids and a mixture thereof.

4. The method of claim 5, wherein the acid is lactic acid.

5. The method of claim 1, wherein the plant comprises recombinant DNA.

6. The method of claim 1, wherein the plant comprises one or more recombinant genes.

7. The method of claim 1, wherein the plant expresses a recombinant protein.

8. The method of claim 1, wherein the plant expresses one or more recombinant materials.

9. The method of claim 10, wherein the recombinant material is a polymer or a macromolecule.

10. The method of claim 1, wherein the saccharifying provides sugars selected from the group consisting of glucose, xylose, arabinose, mannose and galactose.

11. The method of claim 1, wherein the product is selected from the group consisting of pharmaceuticals, nutriceuticals, proteins, fats, vitamins, oils, fiber, minerals, sugars, carbohydrates and alcohols.

12. The method of claim 1, wherein the feedstock is irradiated with a total dose of from about 5 Mrad to about 50 Mrad.

13. The method of claim 1, wherein the feedstock comprises a crop residue.

14. The method of claim 1, wherein the plant is a genetically modified alfalfa, potato, wheat, beet, cotton, rapeseed, rice, or sugarcane plant.
